(19) United States
(12) Patent Application Publication
(10) Pub. No.: US 2012/0058529 A1
(43) Pub. Date: Mar. 8, 2012

(54) METHOD FOR HIGHLY EFFICIENT PRODUCTION OF LACTIC ACID USING CANDIDA UTILIS

(75) Inventors: Shigehito Ikushima, Kanagawa-Ken (JP); Toshio Fujii, Kanagawa-Ken (JP); Osamu Kobayashi, Kanagawa-Ken (JP); Hiroshi Ashigai, Kanagawa-Ken (JP)

(73) Assignee: Kirin Holdings Kabushiki Kaisha

(21) Appl. No.: 13/202,820

(22) PCT Filed: Feb. 23, 2010
(86) PCT No.: PCT/JP2010/052753

§ 371 Date: Nov. 2, 2011

(30) Foreign Application Priority Data
Feb. 23, 2009 (JP) 2009039820

(51) Int. Cl. C12P 7/56; C12N 1/19

(52) U.S. Cl. 435/139; 435/254.22

(57) ABSTRACT

There is disclosed a yeast strain of Candida utilis, wherein the yeast strain has been transformed with at least one copy of a gene that is operatively linked to a promoter sequence enabling expression of the gene and encodes a polypeptide having activity of lactate dehydrogenase. The yeast strain is useful for producing lactic acid with high efficiency.

### REFERENCE TO RELATED APPLICATION

The present patent application claims the priority based on Japanese Patent Application No. 2009-39820 (filed on Feb. 23, 2009) previously filed in Japan and its whole disclosure is incorporated herein by reference.

### BACKGROUND OF THE INVENTION

1. Field of the Invention

The present invention relates to a method of producing lactic acid using Candida utilis, a Crabtree-negative yeast, as a host.

2. Background Art

Biodegradable plastics have recently attracted increased attention due to tackling on environmental problems. Since biodegradable plastics enable recycling of resources to the nature and can be degraded naturally, they pose less burden on the environment. Polylactic acid, a representative raw material of biodegradable plastics, is produced by polymerization of L-lactic acid, and lactic acid with a higher optical purity can provide more stable polylactic acid. Lactic acid is usually obtained as a microbial metabolic product from saccharides such as glucose as a substrate. In particular, a class of bacteria called Lactobacillus has long been known to produce lactic acid specifically and is involved in the production of yogurt, etc. Since Lactobacillus produces D-lactic acid as a byproduct at several percentages in addition to L-lactic acid during a fermentation process, the optical purity of lactic acid produced decreases.

Yeast has often been used in the production of useful substances. Yeast can be cultured generally at a higher cell density than bacteria and can be cultured continuously. Furthermore, yeast secretes proteins in a culture medium, and the secreted proteins are modified by a sugar chain. Production of proteins using yeast is advantageous when such a modification is important for bioactivity.

Yeasts of the genus Saccharomyces have been most studied to date with accumulated genetic findings among yeasts and have been studied as a host for production of various substances.

Furthermore, transformation techniques have been investigated for some yeast species including yeasts of genus Pichia, genus Hansenula, genus Kluyveromyces, and genus Candida in addition to yeasts of genus Saccharomyces and these yeasts are being investigated as a host for production of useful substances. Among them, yeasts of the genus Candida have properties, such as a wide range of carbon assimilation, not owned by the yeasts of genus Saccharomyces.

Among the yeasts of genus Candida, Candida utilis exhibits excellent properties of assimilating pentoses including xylose. In addition, since Candida utilis does not produce ethanol by culturing under aerobic conditions, unlike Saccharomyces yeasts, and its proliferation is thus not inhibited by ethanol, its cells can be produced efficiently by continuous culture at a high density. Accordingly, Candida utilis attracted attention as a protein source and its cells were produced industrially using a saccharified solution or spent sulfite liquor of broadleaf trees which contain a large amount of pentoses as a sugar source. Further, this yeast is approved as a yeast which can be used safe as a food additive together with Saccharomyces cerevisiae and Saccharomyces fragilis by the Food and Drug Administration (FDA) in the U.S. Candida utilis is actually manufactured in various counties worldwide, such as Germany, as well the U.S., Taiwan, and Brazil, and used as food and fodder. In addition to the use as such a microbial protein, Candida utilis has been widely used in the industries as a fermentation strain for pentose and xylose and as a strain for manufacturing ethyl acetate, L-glutamine, glutathione, invertase, and the like.

As an attempt to manufacture lactic acid using yeast, a technique in which a foreign gene encoding a polypeptide having lactate dehydrogenase (LDH) activity is introduced in a yeast having no lactic acid producing ability to produce lactic acid has been developed. Such genetically engineered yeast can produce lactic acid via pyruvic acid from glucose. Since Saccharomyces cerevisiae, the most studied yeast, has a high alcohol fermentation ability to produce ethanol via acetaldehyde from pyruvic acid, its lactic acid production efficiency from a substrate glucose decreases. Then, it has been attempted to disrupt a gene encoding a polypeptide having pyruvate decarboxylase (PDC) activity in the chromosome of Saccharomyces cerevisiae in order to suppress alcohol fermentation (National Publication of International Patent Application No. 2001-516584; National Publication of International Patent Application No. 2003-500062). Saccharomyces cerevisiae has such properties that ethanol fermentation-dependent growth occurs under a high glucose concentration (Crabtree-positive effect), however, and the destruction of the PDC gene thus contributes to the production of lactic acid, but a negative effect of simultaneously suppressing cell proliferation is also induced.

As an example of using a Crabtree effect-negative yeast, on the other hand, production of lactic acid using a yeast strain of genus Kluyveromyces in which at least PDC gene is destroyed has been attempted (National Publication of International Patent Application No. 2001-516584; National Publication of International Patent Application No. 2005-528106); however, the method has disadvantages such as a long fermentation time in a stirred fermentation tank.

In addition, as an example of production of lactic acid using a recombinant yeast of genus Candida, a Crabtree effect-negative yeast, as a host, use of Candida sonorensis is reported (Japanese Patent Application Laid-Open No. 2007-111054; National Publication of International Patent Application No. 2005-518197); however, its lactic acid production efficiency is low, the concentration of lactic acid produced is low, and it takes a long time for production of lactic acid.

Further, there has been no report of highly efficient production of lactic acid using a Crabtree effect-negative lactic-acid producing yeast in a medium containing sucrose, a major constituent sugar of molasses, as a carbon source.

### SUMMARY OF THE INVENTION

The present inventors have found that lactic acid can be more efficiently produced by creating a Candida utilis yeast strain having a gene which encodes a polypeptide having lactate dehydrogenase activity in a expressible manner by transformation technique and culturing the yeast strain. The present invention is based on this finding.

Accordingly, an object of the present invention is to provide a yeast strain producing lactic acid highly efficiently, which strain is created by using Candida utilis, a Crabtree effect-negative yeast, and a method of producing lactic acid at a high yield and a low cost.

The yeast strain according to the present invention is a yeast strain of Candida utilis, wherein the yeast strain has been transformed with at least one copy of a gene that is operatively linked to a promoter sequence enabling expression of the gene and encodes a polypeptide having activity of lactate dehydrogenase.

Further, the method of producing lactic acid according to the present invention comprises culturing the yeast strain according to the present invention.

According to the present invention, a novel Candida utilis strain having an ability of producing lactic acid is provided, and use of this yeast strain for fermentation under appropriate conditions enables production of L-lactic acid efficiently in a short period of time. According to the present invention, the efficiency of lactic acid production can be largely improved while suppressing production of byproducts such as ethanol and various organic acids in the method of producing lactic acid using Candida utilis, a Crabtree effect-negative yeast.

### BRIEF DESCRIPTION OF THE DRAWINGS

FIG. 1 shows the nucleotide sequence (codon-optimized sequence) from a at position 13 to a at position 1011 (TGA in the upstream region among two translation termination codons) of SEQ ID NO: 36 and the alignment of the sequence represented by SEQ ID NO: 38 (bovine-derived wild type sequence).

FIG. 2 shows the structure of the plasmid pCU563.

FIG. 3 shows the structure of the plasmid pCU595.

FIG. 4 shows the annealing site of a primer used for destruction of a CuURA3 gene.

FIG. 5 shows the results of PCR using IM-63 (SEQ ID NO: 58) and IM-92 (SEQ ID NO: 59) as primers. The respective template DNAs are of the NBRC0988 strain (lane 1), the Hygr and G418s strain in which one copy of the CuURA3 gene derived from the NBRC0988 strain is destroyed (lane 2), the Hygs and G418r strain having pCU595 in which one copy of the CuURA3 gene is destroyed (lane 3), and the Hygs and G418s strain in which pCU595 is dropped out and one copy of the CuURA3 gene is destroyed (lane 4). M is a DNA obtained by digestion of Lamda DNA by StyI.

FIG. 6 shows the results of PCR conducted using IM-63 (SEQ ID NO: 58) and IM-223 (SEQ ID NO: 60) as primers. The respective template DNAs are the NBRC0988 strain (lane 1), the Hygr and G418s strain in which one copy of the CuURA3 gene derived from the NBRC0988 strain (lane 2) is disrupted, the Hygs and G418r strain having pCU595 in which one copy of the CuURA3 gene is destroyed (lane 3), and the Hygs and G418s strain in which pCU595 is dropped out and one copy of the CuURA3 gene is destroyed (lane 4). M is a DNA obtained by digestion of Lamda DNA by StyI.

FIG. 7 shows growth abilities of the NBRC0988 strain and the CuURA3 gene-destroyed strain obtained using the NBRC0988 strain as a host in an unselective culture medium and a selective culture medium.

FIG. 8 shows the results of analysis by the Southern hybridization for determining the number of types of PDC gene in Candida utilis. Lane 1 is a sample obtained by digesting a genomic DNA extracted from Saccharomyces cerevisiae S288C by HindIll. Other lanes represent samples obtained by digesting the genomic DNA extracted from the Candida utilis NBRC0988 strain by XbaI (lane 2), HindIII (lane 3), BglII (lane 4), EcoRI (lane 5), BamHI (lane 6), and PstI (lane 7). A DNA fragment of about 220 by (SEQ ID NO: 3), which was obtained by preparing primers IKSM-29 (SEQ ID NO: 1) and IKSM-30 (SEQ ID NO: 2) and amplifying by PCR using a genome of the NBRC0988 strain as a template, was utilized as a probe DNA.

FIG. 9 shows the site of annealing of the primer utilized for destruction of the CuPDC1 gene.

FIG. 10 shows the construction of the plasmid pCU681.

FIG. 11 shows the time-course changes in L-lactic acid concentration in the culture medium from 4 hours to 13 hours after the start of fermentation for the Pj0404 strain and the Pj0957 strain.

FIG. 12 shows the time-course changes in glucose level and L-lactic acid level in the culture medium at multiple samplings up to 24 hours after the start of fermentation in the experiment using calcium carbonate as a neutralizer.

FIG. 13A shows the time-course changes in glucose level and the L-lactic acid level in the culture medium at multiple samplings up to 24 hours after the start of fermentation in the experiment using sodium hydroxide as a neutralizer (n=2).

FIG. 13B shows the time-course changes in glucose level and L-lactic acid level in the culture medium at multiple samplings up to 24 hours after the start of fermentation in the experiment using sodium hydroxide as a neutralizer (n=3).

### DETAILED DESCRIPTION OF THE INVENTION

The yeast, Candida utilis, used in the present invention is produced for food and fodder and is known to be highly safe.

The yeast strain according to the present invention is obtained by transforming a strain of Candida utilis by at least one copy of a gene encoding a polypeptide having lactate dehydrogenase activity, which is operatively linked to a promoter sequence that enables expression of the gene.

Further, analysis using Southern hybridization has indicated that Candida utilis has at least one gene encoding a polypeptide having pyruvate decarboxylase activity (CuPDC1 gene). When the CuPDC1 gene is disrupted, the reaction of converting pyruvic acid into acetaldehyde does not proceed, and thus alcohol fermentation, which is a subsequent step of the metabolic route, does not occur so that ethanol is scarcely produced. In the present invention, when a yeast in which a gene encoding a polypeptide having pyruvate decarboxylase activity is disrupted is used as a host yeast for lactic acid production, ethanol, an excess material, for lactic acid production is not produced and thus lactic acid can be produced with high efficiency.

Accordingly, according to a preferred embodiment of the present invention, a yeast strain with no or reduced pyruvate decarboxylase activity and having a gene encoding a polypeptide having lactate dehydrogenase activity in an expressible manner is provided. It is preferable that an endogenous gene encoding a polypeptide having pyruvate decarboxylase activity is disrupted in this yeast strain.

Further, the gene encoding a polypeptide having lactate dehydrogenase activity is preferably contained in an expressible manner under the regulation of the promoter of a gene encoding a polypeptide having pyruvate decarboxylase activity, more preferably contained in an expressible manner under the regulation of the promoter of a gene encoding a polypeptide having pyruvate decarboxylase activity on the yeast chromosome.

According to a particularly preferred embodiment of the present invention, a yeast strain is provided, which is a yeast producing lactic acid and has a gene encoding a polypeptide having pyruvate decarboxylase activity on the chromosome disrupted and contains a gene encoding a polypeptide having lactate dehydrogenase activity in an expressible manner under the regulation of the promoter of the destroyed gene.

For the yeast strain according to any of these embodiments, the gene encoding a polypeptide having pyruvate decarboxylase activity is preferably a pyruvate decarboxylase gene 1 (CuPDC1 gene), and the polypeptide having lactate dehydrogenase activity is preferably derived from bovine.

Further, the lactic acid produced by the yeast strain according to the present invention may be any of L-lactic acid, D-lactic acid, and DL-lactic acid, but is preferably L-lactic acid.

The yeast strain according to the present invention will be explained below, together with a method of producing lactic acid using the yeast.

Yeast used in the Production of Lactic Acid

The yeast strain according to the present invention is a transformed yeast having a foreign gene encoding a polypeptide having lactate dehydrogenase activity. The yeast used for the production of lactic acid is Candida utilis, a Crabtree-negative yeast. The strain of Candida utilis may be any of various strains known in the art, such as NBRC0626 strain, NBRC0639 strain, NBRC0988 strain, and NBRC1086 strain, but it is preferably NBRC0988 strain.

### Pyruvate Decarboxylase

The yeast strain according to the present invention preferably has no or reduced pyruvate decarboxylase (PDC) activity. This enzyme is an enzyme which converts pyruvic acid into acetaldehyde in the alcohol fermentation cycle, and a yeast involved in alcohol fermentation has intrinsically a gene encoding a polypeptide having pyruvate decarboxylase activity on its chromosome. In Saccharomyces cerevisiae, three genes encoding a polypeptide having pyruvate decarboxylase activity (ScPDC1, ScPDC5 and ScPDC6) are present and these genes function by the so-called autoregulation mechanism. In addition, these genes have high homology to each other of 70% or higher in the nucleotide level. The protein encoded by these genes is composed of the TPP binding region on the N-terminal side and the PDC activity region on the C-terminal side. The genes encoding PDC are present also in other yeasts, and, for example, KIPDC1 gene of Kluyveromyces lactis has high homology to the ScPDC1 gene. On the other hand, Candida utilis has one gene encoding a polypeptide having pyruvate decarboxylase activity (CuPDC1) and alcohol fermentation hardly occurs when at least the CuPDC1 gene is destroyed, although other similar genes may be present.

The expression “(have) no or reduced PDC activity” herein means that the enzyme of interest with no PDC activity or activity lower than that of the wild type is produced, or the amount of production of the enzyme is lower than that of the wild type. The yeast strain having no or reduced PDC activity may be a strain which is obtained by engineering or found by screening. Engineering for such elimination of or reduction in enzyme activity may be conducted by methods known in the art, such as a method using RNAi, a method comprising replacing with another gene such as all or part of the sequence of a selection marker, and a method in which a nonsense sequence is inserted inside a gene. Among others, it is preferable to destroy (knockout) a gene encoding a polypeptide having the activity of the enzyme of interest, and examples of such a method include a method comprising exchanging another gene such as all or part of the sequence of a selection marker with a gene encoding PDC.

The gene encoding a polypeptide having pyruvate decarboxylase activity to be destroyed is originally present in Candida utilis, and the gene described in the Examples of the present invention is one of the allelles of the CuPDC1 gene present in the NBRC0988 strain, whose nucleotide sequence is represented by SEQ ID NO: 63, and the amino acid sequence encoded thereby is represented by SEQ ID NO: 64. When another strain of Candida utilis, such as NBRC0626 strain, NBRC0639 strain, or NBRC1086 strain, is used, any gene having equivalent function, that is, activity, even with a sequence different from the sequence of interest can be destroyed.

According to a preferred embodiment of the present invention, the endogenous gene encoding a polypeptide having pyruvate decarboxylase activity is a gene encoding a polypeptide comprising the amino acid sequence represented by SEQ ID NO: 64, more preferably a gene comprising the nucleotide sequence represented by SEQ ID NO: 63.

### Lactate Dehydrogenase

The yeast strain according to the present invention carries a gene encoding a polypeptide having lactate dehydrogenase activity (LDH gene). Since yeast originally has no lactic acid production ability, the gene encoding a polypeptide having lactate dehydrogenase activity (LDH) which the yeast strain according the present invention carries is a foreign gene. LDH has various analogues depending on the type of organisms or in organisms, and LDH used in the present invention may be either of L-LDH and D-LDH, but is preferably L-LDH. In addition, the gene encoding a polypeptide having lactate dehydrogenase activity used in the present invention includes naturally-occurring LDH as well as artificially synthesized LDHs by chemical synthesis or genetic engineering technique. Examples of the organisms having LDH include prokaryotes such as Lactobacillus, eukaryote such as molds, and higher eukaryotes such as plants, animals, and insects. The LDH used in the present invention is preferably derived from higher eukaryotes and LDH derived from bovine is particularly suitable. The nucleotide sequence of the gene encoding a polypeptide having lactate dehydrogenase (L-LDH) activity derived from bovine is represented by SEQ ID NO: 38, and the amino acid sequence encoded thereby is represented by SEQ ID NO: 35.

According to a preferred embodiment of the present invention, the polypeptide having lactate dehydrogenase activity is a polypeptide comprising the amino acid sequence represented by SEQ ID NO: 37. In addition, the polypeptide having lactate dehydrogenase activity may be a polypeptide comprising an amino acid sequence derived from the amino acid sequence represented by SEQ ID NO: 37 by deletion, substitution, addition or insertion of one or several amino acids, and having lactate dehydrogenase activity.

Here, the deletion, substitution, addition, or insertion of an amino acid(s) can be performed by modifying the gene encoding the above-described polypeptide by a method known in the art. Mutation may be introduced into a gene by a known technique such as the Kunkel method or the Gapped duplex method or a similar method thereto, and for example, using a mutagenesis kit making use of a site-specific mutagenesis method, for example, Mutant-K (Takara Bio Inc.) and Mutant-G (Takara Bio Inc.), or using a LA PCR in vitro Mutagenesis series kit of Takara Bio Inc., or a KOD-Plus-Mutagenesis Kit (TOYOBO). Lactate dehydrogenase activity can be confirmed by techniques known in the art.

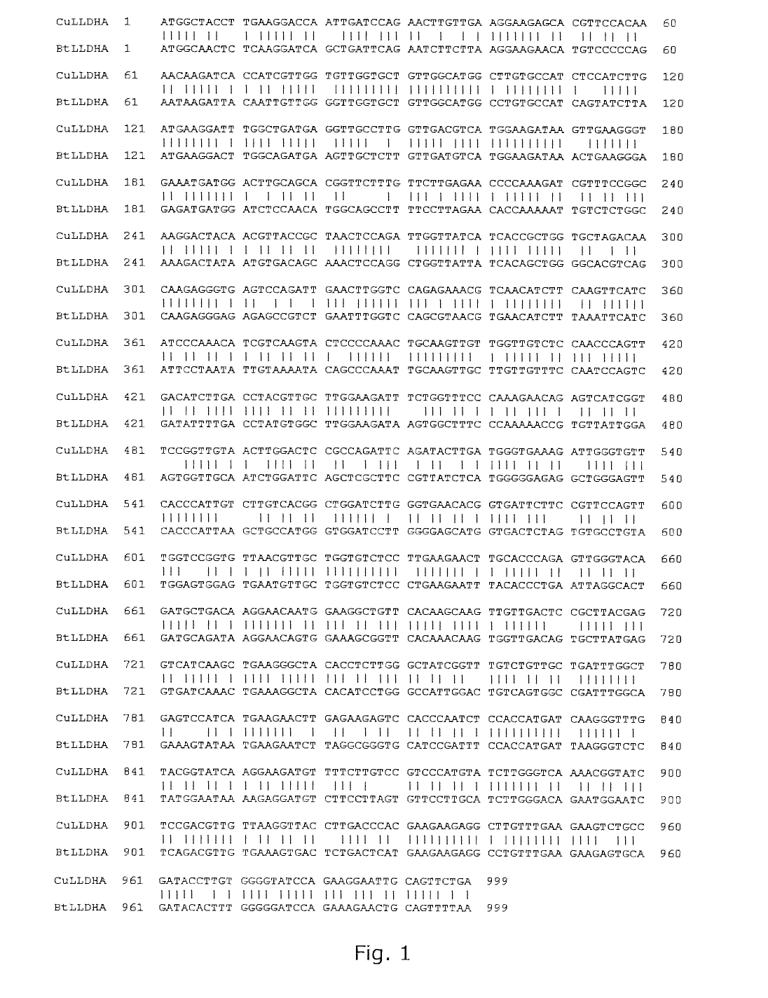

Further, the gene encoding a polypeptide having lactate dehydrogenase activity to be introduced into a host is preferably obtained by artificially synthesizing, considering codon usage frequency of Candida utilis, a nucleotide sequence corresponding to the amino acid sequence (DDBJ/EMBL/GenBank Accession number: AAI46211.1) of the enzyme derived from bovine (Bos taurus) represented in SEQ ID NO: 35. Such artificial synthesis can be performed appropriately by the person skilled in the art, but a particularly preferred nucleotide sequence is a nucleotide sequence comprising a at position 13 to a at position 1,011 of SEQ ID NO: 36. The sequences upstream or downstream thereof are restriction sites, a KpnI recognition site (sequence from g at position 1 to c at position 6 of the nucleotide sequence of SEQ ID NO: 36), an Xba I recognition site (sequence from t at position 7 to a at position 12 of the nucleotide sequence of SEQ ID NO: 36), a BamHI recognition site (sequence from g at position 1,015 to c at position 1,020 of the nucleotide sequence of SEQ ID NO: 36), and a SacI recognition site (sequence from g at position 1,021 to c at position 1,025 of the nucleotide sequence of SEQ ID NO: 36). Of the nucleotide sequence of SEQ ID NO: 36, the alignments of the nucleotide sequence from a at position 13 to a at position 1,011 (tga in the upstream region among 2 translation termination codons) (codon-optimized sequence: SEQ ID NO: 36) and the nucleotide sequence of SEQ ID NO: 38 (a wild type sequence derived from bovine) are shown in FIG. 1. In the two sequences, 751 of the 999 bases are identical with a homology of 75%. In FIG. 1, the upper sequence is the nucleotide sequence from a at position 13 to a at position 1,011 of SEQ ID NO: 36 (tga in the upstream region among 2 translation termination codons). The lower sequence in FIG. 1 is the nucleotide sequence of L-LDH-A gene derived from Bos Taurus represented by SEQ ID NO: 38 (extracted from DDBJ/EMBL/GenBank Accession number: BC146210.1)(the translation product is SEQ ID NO: 35). Since the gene encoding a polypeptide having lactate dehydrogenase activity which has been synthesized artificially is optimized with respect to codon usage frequency for Candida utilis, L-lactic acid in particular can be produced with high efficiency when the gene is transformed into yeast.

According to a preferred embodiment of the present invention, the gene encoding a polypeptide having lactate dehydrogenase activity is a gene comprising the nucleotide sequence from a at position 13 to a at position 1,011 of SEQ ID NO: 36 or an equivalent thereof. The equivalent means a gene in which some of the nucleotide residues are different, provided that the gene has equivalent function to the gene comprising the nucleotide sequence from a at position 13 and a at position 1,011 of SEQ ID NO: 36. Examples of such an equivalent include genes comprising a nucleotide sequence having homology of 70% or higher, preferably 80% or higher, more preferably 85% or higher, further preferably 90% or higher, most preferably 95% or higher to the nucleotide sequence from a at position 13 to a at position 1,011 of SEQ ID NO: 36, and encoding a polypeptide having lactate dehydrogenase activity. Examples of the equivalent include further a gene comprising a nucleotide sequence which hybridizes with the nucleotide sequence from a at position 13 to a at position 1,011 of SEQ ID NO: 36 or a complementary sequence thereof under stringent conditions, and encodes a polypeptide having lactate dehydrogenase activity. Examples of the equivalent further include a gene comprising a nucleotide sequence derived from the nucleotide sequence from a at position 13 to a at position 1,011 of SEQ ID NO: 36 by deletion, substitution, addition, or insertion of one or several nucleotide residues, and encoding a polypeptide having lactate dehydrogenase activity. According to a particularly preferred embodiment of the present invention, the gene encoding a polypeptide having lactate dehydrogenase activity is a gene comprising the nucleotide sequence from a at position 13 to a at position 1,011 of SEQ ID NO: 36.

Here, the deletion, substitution, addition, or insertion of a nucleotide residue(s) can be performed by modifying the gene containing the above-described sequence by a technique known in the art. Mutation may be introduced into a gene by a known technique such as the Kunkel method or the Gapped duplex method or a similar method thereto, and for example, using a mutagenesis kit making use of a site-specific mutagenesis method. Mutation may be introduced using, for example, Mutant-K (Takara Bio Inc.) and Mutant-G (Takara Bio Inc.), or using a LA PCR in vitro Mutagenesis series kit of Takara Bio Inc., or a KOD-Plus-Mutagenesis Kit (TOYOBO). Lactate dehydrogenase activity can be confirmed by techniques known in the art.

The numerical value (%) showing homology is calculated using a program for nucleotide sequence comparison, for example, GENETYX-WIN 7.0.0 and default (initial setting) parameters. In other words, a gene(s) on the yeast chromosome can be substituted by homologous recombination, etc., with a gene encoding a polypeptide having a non-identical but equivalent function, that is, activity. Lactate dehydrogenase activity can be confirmed by a technique known in the art.

The stringent conditions are hybridization conditions in which, for example, Rapid-Hyb Buffer (GE Healthcare Bioscience Inc.) is used, the temperature condition is set at preferably 40 to 70° C., more preferably 60° C., and other conditions are in accordance with the attached protocol. After that, a method generally known by the person skilled in the art is used to perform washing with a solution composed of 2×SSC and 0.1% (w/v) SDS for 5 minutes, followed by washing with a solution composed of 1×SSC and 0.1% (w/v) SDS for 10 minutes, further followed by washing with a solution composed of 0.1×SSC and 0.1% (w/v) SDS for 10 minutes. By setting appropriately conditions such as the temperature condition at the time of hybridization and a salt concentration of a solution used for subsequent washing of a membrane, a DNA comprising a nucleotide sequence having a certain level or higher (any of 70%, 80%, 85%, 90%, and 95%) of homology can be cloned. The gene thus obtained may be substituted by homologous recombination with a gene encoding a polypeptide which is not identical in sequence but has equivalent function, that is, each corresponding activity. The lactate dehydrogenase activity can be confirmed by techniques known in the art.

### Promoter Used for Expression of the Structural Gene

The gene encoding a polypeptide having lactate dehydrogenase activity is preferably contained in an expressible manner under the regulation of the promoter having potent promoter activity. Examples of the promoter for Candida utilis include a promoter of the GAP gene encoding a polypeptide having glyceroaldehyde-3-phosphate dehydrogenase activity, a promoter of the PGK gene encoding a polypeptide having phosphoglycerate kinase, a promoter of the PMA gene encoding a polypeptide having plasma membrane proton ATPase activity of Candida utilis (Japanese Patent Application Laid-Open No. 2003-144185), and the promoter is preferably a promoter of a gene encoding a polypeptide having pyruvate decarboxylase activity 1 (CuPDC1 gene). Among these, the promoter described in the Examples of the present invention is the promoter present in Candida utilis NBRC0988 strain (SEQ ID NO: 3). When the other Candida utilis strains, such as NBRC0626 strain, NBRC0639 strain, and NBRC1086 strain, are used, these strains can be used as they are, if they have equivalent function, that is, activity, even if they are different in sequence (a sequence of another strain). The sequence of another strain can be confirmed by a known method by the person skilled in the art.

The gene encoding a polypeptide having lactate dehydrogenase activity is preferably contained in an expressible manner under the regulation of the promoter of the CuPDC1 gene on the yeast chromosome. Candida utilis used as the host yeast strain according to the present invention is assumed to have at least one PDC gene (CuPDC1 gene). A decrease in pyruvate decarboxylase activity and expression of lactate dehydrogenase activity can be simultaneously achieved effectively by destruction of the CuPDC1 gene regulated by this CuPDC1 gene promoter, wherein the gene encoding a polypeptide having lactate dehydrogenase activity is expressed instead.

According to a preferred embodiment of the present invention, the promoter sequence is the promoter region of the endogenous gene encoding pyruvate decarboxylase, and more preferably that comprising the nucleotide sequence represented by SEQ ID NO: 3. Alternatively, the promoter sequence may be an equivalent in which some of the nucleotide residues are different, provided that the sequence has function equivalent to that comprising the nucleotide sequence represented by SEQ ID NO: 3. Examples of such an equivalent include DNAs having homology of 70% or more, preferably 80% or more, more preferably 85% or more, further preferably 90% or more, most preferably 95% or more to the nucleotide sequence represented by SEQ ID NO: 3, and having promoter activity. Further examples of the equivalent include DNAs hybridizing with the nucleotide sequence represented by SEQ ID NO: 3 or a complementary sequence thereof under stringent conditions, and having promoter activity. Further examples of the equivalent include DNAs comprising a nucleotide sequence derived from the nucleotide sequence represented by SEQ ID NO: 3 by deletion, substitution, addition, or insertion of one or several nucleotide residues, and having promoter activity.

Here, the deletion, substitution, addition, or insertion of a nucleotide residue(s) can be performed by modifying the above-described sequence by techniques known in the art. Mutation may be introduced into a gene by a known technique such as the Kunkel method or the Gapped duplex method or a similar method thereto, and for example, using a mutagenesis kit making use of a site-specific mutagenesis method, for example, Mutant-K (Takara Bio Inc.) and Mutant-G (Takara Bio Inc.), or using a LA PCR in vitro Mutagenesis series kit of Takara Bio Inc., or a KOD-Plus-Mutagenesis Kit (TOYOBO). The promoter activity, that is, transcription activity can be confirmed by techniques known in the art.

The numerical value (%) showing homology is calculated using a program for nucleotide sequence comparison, for example, GENETYX-WIN 7.0.0 and default (initial setting) parameters. In other words, a gene(s) on the yeast chromosome can be substituted by homologous recombination, etc., with a gene encoding a polypeptide having a non-identical but equivalent function, that is, activity. The promoter activity, that is, transcription activity can be confirmed by techniques known in the art.

The stringent conditions are hybridization conditions in which, for example, Rapid-Hyb Buffer (GE Healthcare Bioscience Inc.) is used, the temperature condition is set at preferably 40 to 70° C., more preferably 60° C., and other conditions are in accordance with the attached protocol. After that, a method generally known by the person skilled in the art is used to perform washing with a solution composed of 2×SSC and 0.1% (w/v) SDS for 5 minutes, followed by washing with a solution composed of 1×SSC and 0.1% (w/v) SDS for 10 minutes, further followed by washing with a solution composed of 0.1×SSC and 0.1% (w/v) SDS for 10 minutes. By setting appropriately conditions such as a temperature condition at the time of hybridization and a salt concentration of a solution used for subsequent washing of a membrane, a DNA comprising a nucleotide sequence having a certain level or higher (any of 70%, 80%, 85%, 90%, and 95%) of homology can be cloned. The gene thus obtained may be substituted by homologous recombination with a gene encoding a polypeptide which is not identical in sequence but has equivalent function, that is, activity. The promoter activity, that is, transcription activity can be confirmed by techniques known in the art.

### Molecular Breeding of Yeast Strain

Molecular breeding of the yeast strain according to the present invention can be performed by introducing a gene encoding polypeptides having lactate dehydrogenase activity in an expressible manner in a host yeast. In this case, it is preferable to include disruption of the gene encoding PDC in the host yeast. A DNA construct for PDC disruption contains a gene sequence for homologous recombination to be introduced in a particular gene region to disrupt the gene. The gene sequence for homologous recombination herein refers to a gene sequence homologous to a target region, the PDC gene to be disrupted, or a gene in the vicinity. For example, the gene at the target region can be disrupted by introducing a DNA fragment having, as two gene sequences for homologous recombination, gene sequences homologous to the genes upstream and downstream of the target gene on the chromosome, and a gene for gene disruption provided between these gene sequences for homologous recombination on the yeast chromosome by homologous recombination. Selection of gene sequences for homologous recombination for achieving introduction on a chromosome is known by the person skilled in the art, and the person skilled in the art can construct a DNA sequence for homologous recombination by selecting appropriate gene sequences for homologous recombination, as required.

According to a preferred embodiment of the present invention, the endogenous gene encoding a polypeptide having pyruvate decarboxylase activity is destroyed by deletion of the gene by insertion of a selection marker sequence. For example, the PDC gene can be destroyed by incorporating a selection marker in a nucleotide sequence which is inserted in place of the PDC gene in the homologous recombination. The selection marker is useful for selection of transformed cells. Insertion of the selection marker sequence includes not only introduction of the whole sequence, but also introduction of part of a sequence followed by combining the partial sequence and a sequence originally present in a yeast to complete a selection marker sequence. For example, when a yeast strain lacking part of the selection marker originally present is used as a host for transformation, any gene destruction involving homologous recombination can be performed by introducing the missing part of the sequence as a selection marker. When a yeast is sensitive to drugs such as hygromycin and geneticin (also referred to as G418 hereinbelow), any gene can be destroyed involving homologous recombination by introducing a gene for imparting resistance to these drugs. Thus, according to one embodiment of the present invention, a yeast strain sensitive to the hygromycin B or G418 is used as a host and the PDC gene of the strain is destroyed using a gene imparting a drug resistance originally absent in the host strain. Specific examples of the selection marker include hygromycin B phosphotransferase gene (HPT gene, a gene imparting hygromycin B resistance) and aminoglycoside phosphotransferase (APT gene, a gene imparting G418 resistance), which are shown to be able to be used for the yeast strain in Japanese Patent Application Laid-Open No. 2003-144185.

Although the position on the chromosome where the gene encoding a polypeptide having lactate dehydrogenase activity is incorporated in the yeast genome is not particularly restricted, it is advantageously the gene locus encoding a polypeptide having pyruvate decarboxylase activity. As a result, the gene encoding a polypeptide having lactate dehydrogenase activity can be placed under the regulation of the full-length promoter of the PDC gene, and high expression efficiency can thus be obtained.

According to one embodiment of the present invention, the yeast strain according to the present invention is transformed by an expression vector containing a promoter sequence and a DNA sequence encoding a polypeptide having lactate dehydrogenase activity under the regulation of the promoter sequence. Further, such an expression vector forms one aspect of the present invention.

Candida utilis is hyperploid and does not form spores. When mutation is introduced into a gene of a hyperploid strain, the mutation must be introduced more highly as compared to the case of a monoploid strain. In this case, the possibility that mutation is introduced also into genes other than the target gene of mutation is considered to increase. Accordingly, when a mutation is introduced to the Candida utilis gene, technique that can introduce mutations multiply to a target gene efficiently is preferably used.

Examples of the transformation method of Candida utilis include the technique described in Japanese Patent Application Laid-Open No. 2003-144185. In the above literature, as available vectors, a vector comprising a sequence homologous to the chromosome DNA of Candida utilis and a selection marker and can incorporate a heterogeneous gene into the chromosomal DNA of Candida utilis by homologous recombination, or a vector comprising a DNA sequence having autonomous replication ability in Candida utilis and a selection marker gene and can transform Candida utilis at a high frequency have been developed.

Examples of the selection marker gene used in the transformation system of Candida utilis include a drug-resistance marker which can function in Candida utilis, preferably a cycloheximide-resistant L41 gene, a gene imparting geneticin

(G418) resistance, and a gene imparting hygromycin B resistance. Since the gene imparting geneticin (G418)-resistance and the gene imparting hygromycin B resistance are not present in wild yeast, they are considered to be incorporated in a target gene locus with a high probability. In addition, they are considered to give only a small influence on the trait of a host in other species of yeast. (Baganz F et al., 13 (16): 1563-73.,1997) (Cordero Otero R et al., Appl Microbiol Biotechnol, 46 (2): 143-8, 1996). The gene having these features is considered to be useful for breeding of Candida utilis.

Examples of the other transformation system include a Cre-loxP system derived from bacteriophage P1. This is a system for site-directed recombination between two 34 by loxP sequences, and this recombination is catalyzed by a Cre recombination enzyme encoded by the Cre gene. This system is reported to function also in yeast cells such as Saccharomyces cerevisiae cells, and a selection marker gene placed between two 34 by loxP sequences is known to be removed by recombination between the loxP sequences (Guldener, U. et al., Nucleic Acids Res., 24, 2519-24, 1996). This system is used in a plurality of yeast species such as Kluyveromyces lactis other than Candida utilis (Steensma, H. Y. et al., Yeast, 18, 469-72, 2001).

### Method of Producing Lactic Acid

Lactic acid can be produced in a culture by culturing the yeast strain according to the present invention in the presence of an appropriate carbon source. According to the method of producing lactic acid according to the present invention, lactic acid can be obtained by conducting a step of separating lactic acid from the culture.

Here, the culture in the present invention encompasses, in addition to a culture supernatant, cultured cells and yeast cells and crushed cells and yeast cells.

For culturing the yeast strain according to the present invention, culture methods and culture conditions can be selected depending on the species of yeast. Examples of the culture method include liquid culture using a test tube, flask, or jar fermenter and the mode of culture such as batch culture and semi-batch culture can be adopted. For the culture in a test tube or a flask, a condition of an amplitude of 35 mm is suitable, and such culture can be performed using a bench top cultivator of TAITEC.

In the method of producing lactic acid according to the present invention, the composition of the culture medium is not particularly restricted as far as the composition contains various nutrients which enable yeast growth and production of lactic acid. As the assimilation carbon source contained in the culture medium, xylose and sucrose can be used as far as they can be assimilated, in addition to glucose. According to a preferred embodiment of the present invention, glucose or sucrose is used as the carbon source, more preferably glucose is used.

As the nutrients contained in the culture medium, although yeast extract, peptone, and whey are used, for example, a culture medium containing YP (10 g/L yeast extract and 20 g/L peptone) supplemented with the above-described assimilation carbon source, such as YPD (20 g/L glucose, 10 g/L yeast extract, and 20 g/L peptone), YPX (20 g/L xylose, 10 g/L yeast extract, and 20 g/L peptone), YPSuc10 culture medium (100 g/L sucrose, 10 g/L yeast extract, and 20 g/L peptone), which are further adjusted for pH are convenient.

Here, inorganic nitrogen such as ammonium salts such as ammonium sulfate and urea are more preferably used to prepare a culture medium which is less expensive and has less burden on the purification step. As inorganic nutrient sources, for example, potassium phosphate, magnesium sulfate, and Fe (iron) and Mn (manganese) compounds are also used. The culture medium may further contain a pH adjuster.

The fermentation temperature may be selected in the range where the lactic acid-producing yeast used can be grown. The fermentation temperature may be, for example, about 15° C. to 45°, more preferably 25 to 40° C., further preferably 27 to 40° C., most preferably 35° C. The pH of the culture medium during the fermentation process is preferably maintained at 3 to 8, more preferably 4 to 7, most preferably 6, and lactic acid, a fermentation product, etc., may be neutralized, as required. Examples of the neutralizer used include calcium carbonate, sodium hydroxide, and potassium hydroxide, and the neutralizer is preferably calcium carbonate.

The reaction time required for producing lactic acid is not particularly restricted, and any reaction time can be used as far as the effect of the present invention is achieved. These conditions can be optimized by the person skilled in the art.

When the yeast is first proliferated in the production of lactic acid, it is preferable to conduct pre-preculture and preculture followed by fermentation culture to produce lactic acid.

The conditions of the pre-preculture are as follows: yeast cells grown on YPD agar medium at 30° C. for 1 to 3 days are harvested by scraping with a sterilized toothpick. It is preferable to culture the yeast cells using 3 to 5 mL of the YPD liquid medium charged in a 15-mL tube under the condition of agitation at 120 to 150 rpm.

The conditions of the preculture are as follows: 50 mL to 100 mL of YPD liquid medium, YPX10 liquid medium (100 g/L xylose, 10 g/L yeast extract, 20 g/L peptone) or YPSuc10 liquid medium (100 g/L sucrose, 10 g/L yeast extract, 20 g/L peptone) are used as a culture medium, the yeast cells obtained from the pre-preculture are inoculated to a fresh medium at a density of yeast cells corresponding to the OD600 of about 0.1 and cultured at 120 to 150 rpm, 30° C., and generally for 16 to 30 hours, preferably to the logarithmic growth phase or the stationary phase where the OD600 is 10 to 25.

As for the condition of the fermentation culture, it is preferable to use, as the culture medium, a culture medium containing glucose, xylose or sucrose at a concentration of 95 to 115 g/L (preferably 100 to 115 g/I) and calcium carbonate at a concentration of 3 to 5% as the neutralizer, for example, YPD10 medium (100 g/L glucose, 10 g/L yeast extract, and 20 g/L peptone), YPX10 medium (100 g/L xylose, 10 g/L yeast extract, and 20 g/L peptone) or YPSuc10 culture medium (100 g/L sucrose, 10 g/L yeast extract, and 20 g/L peptone) containing calcium carbonate at 3 to 5%, and to conduct culture at 70 to 150 rpm and 15 to 45° C., with a liquid volume of 10 to 40 mL, under aerated conditions. The culture is conducted more preferably at 80 to 100 rpm and 25 to 40° C. with a liquid volume of 10 to 20 mL, further preferably at 80 rpm and 27 to 40° C. with a liquid volume 10 to 15 mL. It is preferable to use a 100 mL baffled Erlenmeyer flask to which 10 to 40 mL of a culture medium and yeast cells are charged. Particularly in this stage, it is preferable to adjust the initial amount of yeast cells to be the amount corresponding to the OD600 of 1 to 30 (preferably 1 to 20), because a metabolic product (for example, lactic acid) can be produced more efficiently in a short period of time, and the amount is more preferably adjusted to OD600 of 5 to 25, further preferably to OD600 of 5 to 15, most preferably to OD600 of about 10.

In the production of lactic acid in a culture medium scale of 500 mL or more, when yeast is proliferated, the yeast is preferably subjected to pre-pre-preculture, pre-preculture, and preculture in a liquid medium followed by fermentation culture to produce lactic acid. In the test of such a scale, it is preferable to use a jar fermenter.

The conditions of the pre-pre-preculture are as follows: yeast cells grown on YPD agar medium at 30° C. for 1 to 3 days are harvested by scraping with a sterilized toothpick. It is preferable to culture the yeast cells using 3 to 5 mL of YPD liquid medium charged in a 15-mL tube at 120 to 150 rpm and 30° C. generally for 6 to 30 hours under agitating condition.

As for the condition of the pre-preculture, it is preferable to use 50 mL to 100 mL of YPD liquid medium, inoculate the yeast cells from the pre-pre-preculture to a fresh culture medium to the OD600 of about 0.1, and conduct culture at 120 to 150 rpm and 30° C. for generally 10 to 30 hours to the logarithmic growth phase or the stationary phase where the OD600 is 10 to 25. It is preferable to use a Sakaguchi flask for this culture.

As the condition of the preculture, it is preferable to use a jar fermenter for which a temperature, an aeration volume, an agitation speed, etc., can be adjusted. It is preferable to use 500 mL to 2.5 L of YPD liquid medium as the culture medium, add 20 to 100 mL of the pre-preculture to a fresh culture medium for inoculation to the OD600 of about 0.1, and conduct culture at an agitation speed of 300 to 400 rpm at a temperature of 30° C. with an aeration volume of 1.25 vvm for generally 10 to 30 hours to the logarithmic growth phase or the stationary phase where the OD600 is 10 to 25.

As for the condition of the fermentation culture, it is preferable to use jar fermenter for which a temperature, an aeration volume, an agitation speed, pH, etc., can be adjusted. It is preferable to use, as the culture medium, YPD10 medium (100 g/L glucose, 10 g/L yeast extract, and 20 g/L peptone) or YPSuc10 medium (100 g/L sucrose, 10 g/L yeast extract, and 20 g/L peptone) containing glucose or sucrose at a concentration of 50 to 220 g/L, preferably 100 to 115 g/L and calcium carbonate at 3 to 5% as the neutralizer, or YPD10 medium or YPSuc10 medium whose pH is maintained at a pH suitable for fermentation using a neutralizer such as sodium hydroxide and potassium hydroxide and conduct culture at an agitation speed of 100 to 300 rpm at 15 to 45° C. with a liquid volume of 500 mL to 2.5 L. The conditions are more preferably 200 to 250 rpm, 27 to 37° C., with a liquid volume of 1.5 to 2 L. In particular in this stage, it is preferable to adjust the initial amount of yeast cells to be the amount corresponding to the OD600 of 1 to 30 (preferably 1 to 20), because lactic acid can be produced more efficiently in a short period of time, and the amount is more preferably adjusted to the OD600 of 5 to 10 and most preferably adjusted to the OD600 of 5 to 15 and most preferably adjusted to the OD600 of about 10.

The aeration condition during fermentation herein is preferably aerobic condition, especially slightly aerobic condition. Lactic acid is produced highly efficiently by culturing generally for 24 to 48 hours.

In the method of producing lactic acid according to the present invention, the lactic acid component thus produced is separated from the culture medium and recovered. According to the method of producing lactic acid according to the present invention, known methods used in the conventional production process by lactic acid fermentation can be used for the aeration and condensation means for the lactic acid component. Examples of these known methods include, for example, (1) recrystallization of calcium lactate by neutralization with addition of lime milk, (2) organic solvent extraction using a solvent such as ether, (3) esterification separation by esterifying purified lactic acid with alcohol, (4) chromatographic separation using an ion chromatographic resin, and (5) electrodialysis using an ion-exchange membrane. Accordingly, the lactic acid component obtained by the method of producing lactic acid according to the present invention may be not only free lactic acid but also in the form of a salt with sodium, potassium, or the like and an ester such as methyl ester and ethyl ester.

According to the method of producing lactic acid according to the present invention, the ability of the yeast Candida utilis of producing its metabolic product (for example, lactic acid) in the yeast having the production ability can be improved, and as a result, lactic acid can be produced highly efficiently in a short period of time. The method of producing lactic acid according to the present invention can improve lactic acid producing ability of a yeast having the ability of producing lactic acid, regardless of the composition of the culture medium used. Consequently, according to the method of producing lactic acid according to the present invention, the ability of producing lactic acid can be improved, even with a culture medium with less nutrient, such as a relatively cheap synthetic culture medium, and thus the production cost for lactic acid can be reduced.

In particular, according to the method of producing lactic acid according to the present invention, when a yeast having also an ethanol production ability, such as yeasts to which the gene encoding a polypeptide having lactate dehydrogenase activity has been introduced, production of ethanol can be suppressed and lactic acid can be produced highly efficiently. In addition, lactic acid contained in the culture medium can be recovered more easily by suppressing production of byproducts other than ethanol, such as various organic acids such as D-lactic acid. In other words, the steps involved in recovery and purification of lactic acid can be simplified, and the cost required for the production of lactic acid can be suppressed. These byproducts can be analyzed and evaluated by the known techniques. For example, ethanol can be analyzed and evaluated by gas chromatography (GC) or high performance chromatography (HPLC); aroma components such as acetaldehyde by GC; and organic acids such as pyruvic acid by HPLC. The amount of glucose can be analyzed and evaluated with HPLC or a biochemistry analyzer (BA hereinbelow) (YSI Japan Ltd.); L-lactic acid by HPLC or BA; D-lactic acid by HPLC, or D-lactic acid together with L-lactic acid by an F-kit D-lactic acid/L-lactic acid (J.K. International Inc.). It is preferable to remove, from samples subjected to various analyses, contaminants which may impart adverse influence on analysis, such as stacking of a column, by filtering through a 0.22 μm filter.

Various organic acids such as pyruvic acid, citric acid, malic aid, and succinic acid in the liquid culture were measured by organic acid analysis by HPLC (detection based on electric conductivity). Further, methods of measuring other substances such as ethanol are described in the following Examples.

In the present specification, it has been further found that a large amount of pyruvic acid is produced by culturing a Candida utilis yeast strain in which an endogenous gene encoding a polypeptide having pyruvate decarboxylase activity is destroyed and to which a gene encoding a polypeptide having lactate dehydrogenase activity has not been introduced.

Accordingly, another aspect of the present invention provides a Candida utilis yeast strain in which an endogenous gene encoding a polypeptide having pyruvate decarboxylase activity is destroyed is provided and further a method of producing pyruvic acid comprising culturing the yeast strain. Since pyruvic acid is highly reactive and used as a substrate for synthesis of pharmaceuticals and agricultural chemicals, it is an important intermediate in the field of fine chemicals.

The details of the endogenous gene encoding a polypeptide having pyruvate decarboxylase activity in the Candida utilis yeast strain and its destruction are as described above. As the purification method of pyruvic acid, any method known as a purification method of organic compounds can be used, and, for example, a distillation method described in Japanese Patent Application Laid-Open No. 2007-169244 and the like can be used. Distillation can be performed under reduced pressure or vacuum at 70 to 80° C. for the first distillation and under reduced pressure or vacuum at 90 to 100° C. for the second distillation. Pyruvic acid obtained by the distillation can be separated from products such as lactic acid and recovered by further optionally subjecting to treatment such as treatment with activated carbon, dehydration, and acetic acid removal. Pyruvic acid thus purified can be utilized in the field of fine chemicals, as described above.

### EXAMPLES

Specific examples of the present invention described below are set forth without imposing any limitations upon the technical scope of the present invention.

Unless otherwise stated, Ex Taq from TaKaRa or KOD-Plus-from Toyobo was used for gene amplification by PCR, and the procedure was carried out according to the attached protocol.

After a heat treatment at 94° C. for 1 minute, a PCR amplification reaction cycle consisting of the following three steps was repeated thirty times: the denaturing step at 94° C. for 30 seconds; the annealing step at X° C. for 30 seconds (X° C. is the Tm of primers, though 55° C. was employed unless otherwise specified); and the extension step at 72° C. for Y seconds (Y seconds were calculated from the expected size of the amplification product based on about 60 seconds per kbp (kilo base pair)), with a final temperature of 4° C. As a PCR amplification apparatus, GeneAmp PCR System 9700 (PE Applied Biosystems) was used. For extraction of genomic DNA from yeast, Dr. GenTLE from TaKaRa or the potassium acetate method (Methods Enzymol., 65, 404, 1980) was used.

Alkaline Phosphatase (E. coli C75) from TaKaRa or Alkaline Phosphatase (Shrimp) from TaKaRa was used for dephosphorylation reaction of DNA, and Ligation Kit ver.2 from TaKaRa was used for ligation reaction. The procedures were carried out according to the attached protocols. Competent cells of DH5a (Toyobo) were used for transformation of E. coli, and the procedure was carried out according to the attached protocol. For selection of transformants of E. coli, LB plates containing 100 μg/mL Ampicillin (LB+amp plates) or LB plates containing 50 μg/mL kanamycin were used, depending on the drug resistance marker gene contained in a plasmid, and, if necessary, blue-white selection with 20 μg/mL X-gal and 0.1 mM IPTG was performed. QIAprep Spin Miniprep Kit from QIAGEN was used to recover plasmid DNA from E. coli, and the procedure was carried out according to the attached protocol. Transformation of Saccharomyces cerevisiae was performed by the lithium method (Ito et al., J. Bacteriol., 153, 163, 1983). Transformation of Candida utilis was performed according to the method described in Japanese Patent Application Laid-Open No. 2003-144185 with some modifications. Determination of base sequences was performed according to the following method. BigDye Terminator v3.1 from Applied Biosystems was used to perform PCR, and the procedure was carried out according to the attached protocol. CENTRI-SEP COLUMNS (PRINCETON SEPARATIONS) was used to remove unreacted BigDye Terminator, and the procedure was carried out according to the attached protocol. 3100 Genetic Analyzer from Applied Biosystems was used to determine base sequences, and the procedure was carried out according to the attached protocol. Note that in representations of degenerate primers provided in the sequence listing, “W” represents a mixture of “A (adenine)” and “T (thymine),” “R” represents a mixture of “A (adenine)” and “G (guanine),” “Y” represents a mixture of “C (cytosine)” and “T (thymine),” and “M” represents a mixture of “A (adenine)” and “C (cytosine).” In addition, the numerical values in tables, such as the amount of lactic acid production, are presented as mean±standard error of the mean.

Transformation of Candida utilis strains using electric pulses was performed according to the method described in Japanese Patent Laid-Open Publication No. 2003-144185 with some modifications. The colony on a YPD plate is cultured in 5 mL of YPD liquid medium with shaking at 30° C. for about 8 hours, then inoculated into 200 mL of YPD liquid medium to an OD600 of 0.0024, and cultured with shaking at 30° C. After about 16 hours, the cells are collected by centrifugation at 1,400×g for 5 minutes after having grown to logarithmic growth phase (OD600=2.5). The cells are washed once with 100 mL of ice-cold sterile water, then once with 40 mL of ice-cold sterile water, and subsequently once with 40 mL of ice-cold 1 M sorbitol. After suspended in 10 mL of 1 M sorbitol, the cells are transferred to a sterile polypropylene tube and again collected by centrifugation at 1,100×g for 5 minutes. After removal of the supernatant, the cells are suspended in ice-cold 1 M sorbitol to bring the final volume of the cell solution to 2.5 mL.

A transformation experiment using electric pulses is performed with Gene Pulser from Bio-Rad. After 50 μL of the cell solution is mixed with 5 μL of a DNA sample containing 100 ng to 10 μg of DNA and with 5 μL of 2.0 mg/mL carrier DNA from salmon testes, the mixture is placed in a 0.2 cm disposable cuvette, and electric pulses of appropriate conditions are applied to it. For example, according to a preferred aspect of the present invention, the pulses are applied under the conditions of a capacitance of 25 μF, a resistance value of from 600 to 1000 ohms, and a voltage of from 0.75 to 5 KV/cm. After the pulse application, 1 mL of an ice-cold YPD medium containing 1 M sorbitol was added, and the mixture was transferred to a sterile polypropylene tube, followed by culture with shaking at 30° C. for about 6 to 15 hours. After the culture, the cell solution was plated onto YPD selective medium containing an appropriate agent depending on the selectable marker gene, and then the plate was incubated at 28 to 30° C. for 3 to 4 days to obtain transformant colonies. In the case of using the HPT gene as a selectable marker gene, hygromycin B was added to YPD medium at a concentration of from 600 to 800 μg/mL, and, in the case of using the APT gene as a selectable marker gene, G418 was added to YPD medium at a concentration of 200 μg/mL. Hereinafter, these media are respectively referred to as HygB medium and G418 medium. In addition, being resistant to hygromycin B is expressed as HygBr, being sensitive to hygromycin B is expressed as HygBs, being resistant to G418 is expressed as G418r, and being sensitive to G418 is expressed as G418s.

### Example 1

### Development of a Candida utilis Transformation System which Utilizes the Cre-loxP System

1-1. Construction of a Plasmid Required in a Multiple Transformation System which Utilizes the Cre-lox System

pCU563, a plasmid to prepare a DNA fragment for gene disruption, was constructed according to the following procedure. Using as a template the plasmid pGKHPT1 described in Shimada et al. (Appl. Environ. Microbiol. 64, 2676-2680), which carries the PGK gene promoter and the HPT gene (hygromycin-resistant gene), PCR (extension reaction: 1.5 minutes) was performed with a primer set of IM-53 (SEQ ID NO:16) and IM-57 (SEQ ID NO:17) to amplify a DNA fragment consisting of, in sequence, loxP (SEQ ID NO:18), the PGK gene promoter, and the HPT gene. In addition, using pGAPPT10 (Kondo et al., Nat. Biotechnol. 15, 453-457) as a template, PCR (extension reaction: 30 seconds) was performed with a primer set of IM-54 (SEQ ID NO:19) and IM-55 (SEQ ID NO:20) to amplify a DNA fragment that consists of the GAP gene terminator and loxP.

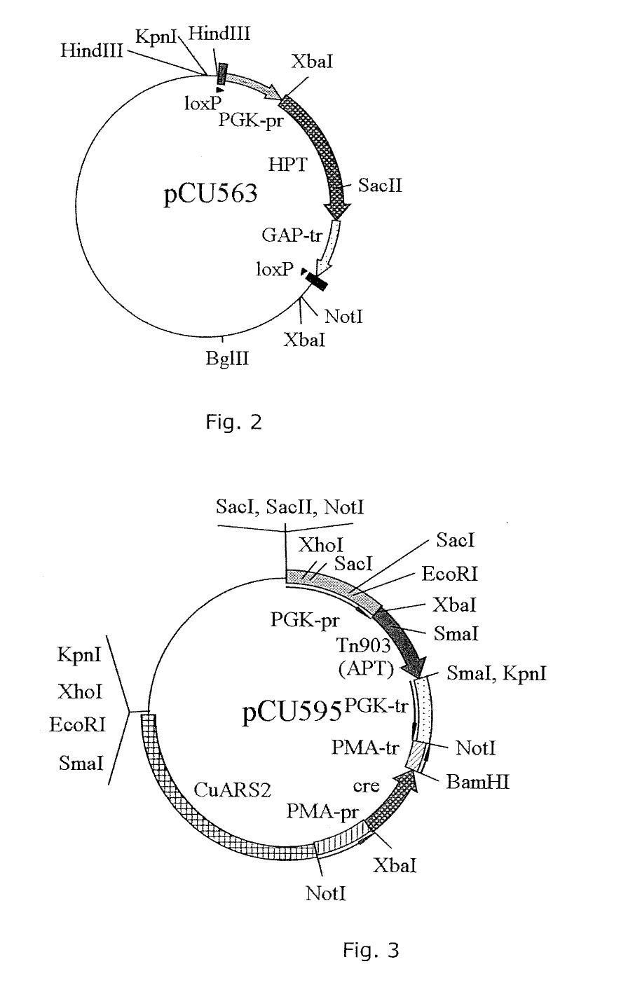

These were mixed together and subjected to PCR (extension reaction: 2 minutes) with IM-1 (SEQ ID NO:21) and IM-2 (SEQ ID NO:22) to amplify a DNA fragment consisting of, in sequence, loxP, the PGK gene promoter, the HPT gene, the GAP gene terminator, and loxP. The resulting DNA fragment was cloned into a pCR2.1 vector [Invitrogen: TA cloning kit (pCR2.1 vector)]. The plasmid thus obtained was designated pCU563 (FIG. 2). Incorporation of this module into Candida utilis cells by transformation enables the cells to grow in a medium containing HygB at a concentration of from 600 to 800 μg/mL, where wild-type strains, for example, cannot grow.

pCU595, an expression plasmid for the Cre recombinase, was constructed according to the following procedure. Using the plasmid pSH65 for expressing Cre in S. cerevisiae (Gueldener, U. et al., Nucleic Acids Res. 30(6), E23, 2002) as a template, PCRs were performed with two primer sets of (1) IM-49 (SEQ ID NO:23) and IM-50 (SEQ ID NO:24), and of (2) IM-51 (SEQ ID NO:25) and IM-52 (SEQ ID NO:26) (extension reaction of each PCR: 30 seconds). The respective amplified DNA fragments were mixed together and then subjected to PCR with IM-49 (SEQ ID NO:23) and IM-52 (SEQ ID NO:26) to amplify a gene segment encoding the Cre recombinase. In the Cre gene thus produced, the BamHI recognition sequence (GGATCC) present in the Cre gene of pSH65 has been altered, with no change in amino acid sequence, to be a sequence (GCATAC) that is not recognized by the BamHI enzyme. In addition, a DNA fragment obtained by digesting the gene with XbaI and BamHI was inserted into the XbaI-BamHI gap of pPMAPT1 (Japanese Patent Application Laid-Open No. 2003-144185). The Cre expression module obtained by treating this plasmid with NotI, i.e., a DNA fragment consisting of, in sequence, the PMA gene promoter, the Cre gene, and the PMA gene terminator, was inserted into DNA obtained by partially digesting pCARS7 (Japanese Patent Application Laid-Open No. 2003-144185), having the autonomously replicating sequence CuARS2, with NotI. The plasmid thus obtained was designated pCU595 (FIG. 3). This plasmid contains the APT gene, and if transformation of Candida utilis with this plasmid is performed, the cells into which the plasmid has been introduced become able to grow in a medium containing G418 at a concentration of 200 μg/mL, where wild-type strains, for example, cannot grow.

1-2. Multiple Disruptions of the CuURA3 Genes through the use of the Cre-lox System

To see whether the Cre-loxP system functions or not, an attempt was made to make multiple disruptions of the Candida utilis URA3 genes (hereinafter referred to as the CuURA3 genes) described in Japanese Patent Application Laid-Open No. 2003-144185. These genes encode the orotidine-5′-phosphate decarboxylase, and a strain that has lost all these functional genes in the cell will become auxotrophic for uracil. That is, it is considered that the strain will become unable to grow in uracil-free medium.

A DNA fragment to disrupt the first and second copies of the CuURA3 gene was prepared as follows. First, the following two PCRs shown in (1), (2), and (3) were performed: (1) pCU563 was used as a template, IM-1 (SEQ ID NO:21) and IM-2 (SEQ ID NO:22) as primers, and the extension reaction time was 2 minutes; (2) genomic DNA from the NBRC 0988 strain was used as a template, IM-59 (SEQ ID NO:54) and IM-60 (SEQ ID NO:55) as primers, and the extension reaction time was 30 seconds; and (3) genomic DNA from the NBRC 0988 strain was used as a template, IM-61 (SEQ ID NO:56) and IM-62 (SEQ ID NO:57) as primers, and the extension reaction time was 30 seconds. In (2) and (3), the upstream portion and downstream portion of the CuURA3 gene are amplified. Further, the following PCR in (4) was performed: (4) a mixture of the three DNAs amplified in (1), (2), and (3) above was used as a template, IM-59 (SEQ ID NO:54) and IM-62 (SEQ ID NO:57) as primers, and the extension reaction time was 3 minutes. This provided a DNA fragment consisting of, in sequence, the upstream region of the CuURA3 gene, loxP, the PGK gene promoter, the HPT gene, the GAP gene promoter, loxP, and the downstream region of the CuURA3 gene. Hereinafter, this DNA fragment is referred to as “the first/second CuURA3 disruption fragment.”

Transformation with this DNA fragment causes double-strand homologous recombination to occur in the upstream region and downstream region of the CuURA3 gene, and thereby makes it possible to partially delete an allele of the CuURA3 gene.

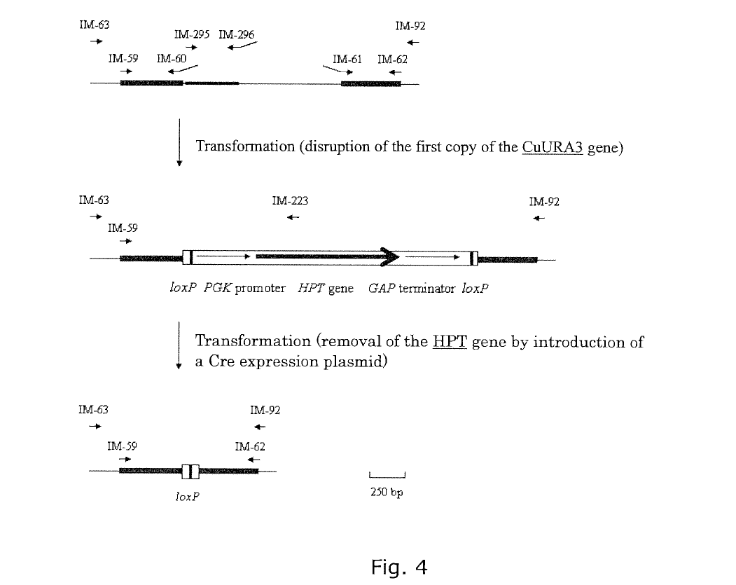

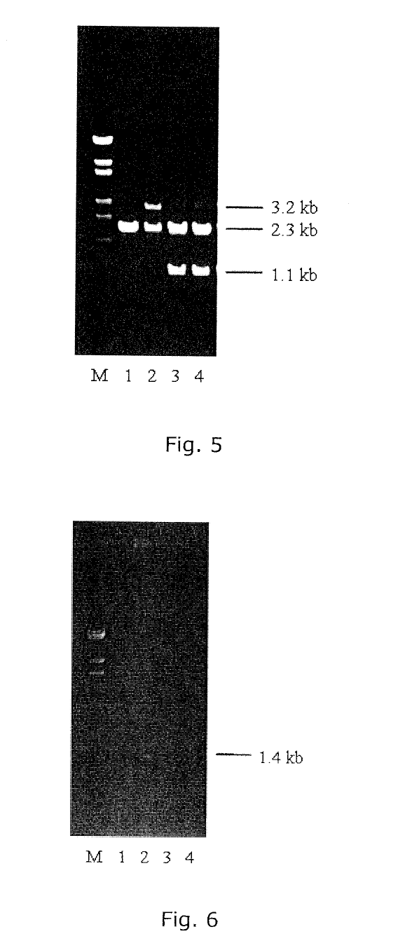

Using 1 pg of the first/second CuURA3 disruption fragment as a DNA fragment, transformation of the NBRC 0988 strain was performed. As a result, 119 clones of HygBr transformants were obtained. Genomic DNA was extracted from the NBRC 0988 strain and 11 clones of transformants randomly selected from the 119 clones, and used as a template to perform PCR with IM-63 (SEQ ID NO:58) and IM-92 (SEQ ID NO:59) (extension reaction: 3.5 minutes). As shown in FIG. 4, these primers anneal outside the homologous recombination area. When the resultant products were subjected to 0.8% agarose gel electrophoresis, it was found that a 2.3 kb DNA fragment was amplified from the NBRC 0988 strain, and 3.2 kb and 2.3 kb DNA fragments were amplified from all the 11 clones of transformants (FIG. 5). This proved that a strain of interest in which one CuURA3 gene copy was disrupted was obtained in the HygBr. In addition, it was also revealed that there is a high probability that positive clones can be selected from the transformants.

The HygBr strain in which one CuURA3 gene copy was disrupted was transformed with the Cre expression plasmid pCU595. In G418-containing medium, the negative control to which DNA was not added did not form a colony, while 1,000 or more transformants were obtained from the sample to which pCU595 was added. Thirty strains of them were plated onto G418 medium or HygB medium, and the results were that they were all able to grow on the G418 medium but were not able to grow on the HygB medium.

Genomic DNAs were extracted from the HygBr strain in which one CuURA3 gene copy was disrupted and from the HygBs strain in which first CuURA3 gene copy was disrupted, and used as templates to perform PCRs with IM-63 (SEQ ID NO:58) and IM-92 (SEQ ID NO:59) (extension reaction: 3.5 minutes). When the resultant products were subjected to 0.8% agarose gel electrophoresis, it was found that 3.2 kb and 2.3 kb DNA fragments were amplified from the former strain, and 2.3 kb and 1.1 kb DNA fragments were amplified from the latter strain. A strain in which the HPT gene has been eliminated as intended was obtained.

After the HygBs strain in which one CuURA3 gene copy was disrupted was cultured overnight in YPD liquid medium, a portion of the culture was plated onto YPD medium. After 1 to 3 days, a plurality of single colonies were separated and plated onto a G418 medium and YPD medium. The results were that most clones grew on the YPD medium but did not grow on the G418 medium.

FIG. 5 shows the results of PCRs performed with IM-63 (SEQ ID NO: 58) and IM-92 (SEQ ID NO:59) as primers, using as templates, in turn, DNAs extracted from the NBRC 0988 strain, from the Hygr and G418s strain in which one copy of the CuURA3 gene derived from the NBRC 0988 strain has been disrupted, from the Hygs and G418r strain constructed by introducing a Cre expression plasmid, and from the Hygs and G418s strain in which the Cre expression plasmid has been omitted (extension reaction: 3.5 minutes). The results of the PCRs respectively correspond to Lane 1, Lane 2, Lane 3, and Lane 4, in this order.

FIG. 6 shows the results of PCRs performed with IM-63 (SEQ ID NO: 58) and IM-223 (SEQ ID NO:60) as plasmids, using as templates, in turn, DNAs extracted from the NBRC 0988 strain, from the Hygr and G418s strain in which one copy of the CuURA3 gene derived from the NBRC 0988 strain has been disrupted, from the Hygs and G418r strain constructed by introducing a Cre expression plasmid, and from the Hygs and G418s strain in which the Cre expression plasmid has been omitted (extension reaction: 2 minutes).

The results of the PCRs respectively correspond to Lane 1, Lane 2, Lane 3, and Lane 4, in this order. As shown in FIG. 4, IM-63 (SEQ ID NO:58) anneals outside of the homologous recombination area, and IM-223 (nSEQ ID NO: 60) anneals inside the HPT gene. The result that a 1.4 kb DNA fragment was amplified only in Lane 2, where the Hygr strain was used as a template, proved that the Cre-loxP system functions also in Candida utilis, similar to the results with IM-63 (SEQ ID NO:58) and IM-92 (SEQ ID NO:59).

Genomic DNAs were extracted from the HygBs G418r strain and the HygBs G418s strain, which both have one copy of the CuURA3 gene disrupted but which are different in the ability to grow in C418-containing medium, and used as templates to perform PCRs with IM-49 (SEQ ID NO:23) and IM-52 (SEQ ID NO:26), a primer set to amplify the Cre gene (extension reaction: 1 minute). As a result, a 1 kb DNA fragment was amplified from the G418r strain but was not amplified from the G418s strain. This confirmed that a HygBs and G418s strain in which first CuURA3 gene copy was disrupted and pCU595 has been omitted was obtained.

Using the first/second CuURA3 disruption fragment as a DNA fragment, transformation of the HygBs and G418s, one CuURA3 gene copy disrupted strain was performed. Genomic DNA was extracted from the resulting transformants and used as a template to perform PCR with IM-63 (SEQ ID NO:58) and IM-92 (SEQ ID NO:59) (extension reaction: 3.5 minutes). When the resultant products were subjected to 0.8% agarose gel electrophoresis, it was found that there was a plurality of strains from which three DNA fragments, 3.2 kb, 2.3 kb, and 1.1 kb, were amplified. This proved that a strain of interest is the HygBr in which two CuURA3 gene copies were disrupted was obtained.

The HygBr strain in which two CuURA3 gene copies were disrupted was transformed with the Cre expression plasmid pCU595. When the resulting transformants were plated onto G418 medium and HygB medium, they were all able to grow on the G418 medium but were not able to grow on the HygB medium. Using genomic DNA extracted from the HygBs and G418r transformant as a template, PCR was performed with IM-63 (SEQ ID NO:58) and IM-92 (SEQ ID NO:59) (extension reaction: 3.5 minutes). When the resultant products were subjected to 0.8% agarose gel electrophoresis, it was found that two DNA fragments, 2.3 kb and 1.1 kb, were amplified. This proved that the HPT gene could be eliminated.

After the HygBs strain in which two CuURA3 gene copies were disrupted was cultured overnight in YPD liquid medium, a portion of the culture was plated onto YPD medium. After two days, single colonies were separated and plated onto YPD medium and G418 medium. Then, clones which grew on the YPD medium but did not grow on the G418 medium were separated. A strain in which pCU595 has been omitted, i.e., the HygBs and G418s strain in which second CuURA3 gene copy was disrupted was obtained.

A DNA fragment to disrupt the third and forth copies of the CuURA3 gene was prepared as follows. First, the following three PCRs shown in (1), (2), and (3) were performed: (1) pCU563 was used as a template, IM-1 (SEQ ID NO:21) and IM-2 (SEQ ID NO:22) as primers, and the extension reaction time was 2 minutes; (2) genomic DNA from the NBRC 0988 strain was used as a template, IM-295 (SEQ ID NO:61) and IM-296 (SEQ ID NO:62) as primers, and the extension reaction time was 30 seconds; and (3) genomic DNA from the NBRC 0988 strain was used as a template, IM-61 (SEQ ID NO:56) and IM-62 (SEQ ID NO:57) as primers, and the extension reaction time was 30 seconds. In (2) and (3), the upstream portion and downstream portion of the CuURA3 gene are amplified. Further, the following PCR in (4) was performed: (4) a mixture of the three DNAs amplified in (1), (2), and (3) above was used as a template, IM-295 (SEQ ID NO:61) and IM-62 (SEQ ID NO:57) as primers, and the extension reaction time was 3 minutes. This provided a DNA fragment consisting of, in sequence, the upstream region of the CuURA3 gene, loxP, the PGK gene promoter, the HPT gene, the GAP gene terminator, loxP, and the downstream region of the CuURA3 gene. Hereinafter, this DNA fragment is referred to as “the third/fourth CuURA3 disruption fragment.” Transformation with this DNA fragment causes double-strand homologous recombination to occur in the upstream region and downstream region of the CuURA3 gene, and thereby makes it possible to partially delete an allele of the CuURA3 gene. In addition, since the upstream region of the CuURA3 gene amplified in (3) is a region deleted in the transformations performed using the first/second CuURA3 disruption fragment to disrupt the first and second copies of the CuURA3 gene, it is conceivable that the possibility of incorporation into the two disrupted copies of the allele can be reduced.

Using the third/fourth CuURA3 disruption fragment as a DNA fragment, transformation of the HygBs and G418s strain in which two CuURA3 gene copies were disrupted was performed to disrupt the third copy of the CuURA3 gene. Genomic DNA was extracted from the resulting transformants and used as a template to perform PCR with IM-63 (SEQ ID NO:58) and IM-92 (SEQ ID NO:59) (extension reaction: 3.5 minutes). When the resultant products were subjected to 0.8% agarose gel electrophoresis, it was found that there were strains from which three DNA fragments, 3.6 kb, 2.3 kb, and 1.1 kb, were amplified. This proved that a strain of interest in the HygBr in which third CuURA3 gene copy was disrupted was obtained.

The HygBr strain in which three CuURA3 gene copies were disrupted was transformed with the Cre expression plasmid pCU595. The resulting transformants were plated onto G418 medium and HygB medium, and the results were that they were all able to grow on the G418 medium but were not able to grow on the HygB medium. Using genomic DNA extracted from the HygBs and the G418r transformant as a template, PCR was performed with IM-63 (SEQ ID NO:58) and IM-92 (SEQ ID NO:59) (extension reaction: 3.5 minutes). When the resultant products were subjected to 0.8% agarose gel electrophoresis, it was found that three DNA fragments, 2.3 kb, 1.5 kb, and 1.1 kb, were amplified. This proved that the HPT gene was eliminated.

After the HygBs strain in which three CuURA3 gene copies were disrupted was cultured overnight in YPD liquid medium, a portion of the culture was plated onto YPD medium. After two days, single colonies were separated and plated onto YPD medium and G418 medium. Then, clones which grew on the YPD medium but did not grow on the G418 medium were separated. A strain in which pCU595 has been omitted, i.e., the HygBs and G418s strain in which third CuURA3 gene copy was disrupted was obtained.

Using the third/fourth CuURA3 disruption fragment as a DNA fragment, transformation of the HygBs and G418s strain in which three CuURA3 gene copies were disrupted was performed. Genomic DNA was extracted from the resulting transformants and used as a template to perform PCR with IM-63 (SEQ ID NO:58) and IM-92 (SEQ ID NO:59) (extension reaction: 3.5 minutes). When the resultant products were subjected to 0.8% agarose gel electrophoresis, it was found that there was a plurality of transformants from which three DNA fragments, 3.6 kb, 1.5 kb, and 1.1 kb, were amplified. This proved that a strain of interest of the HygBr in which four CuURA3 gene copies were disrupted was obtained. In addition, since the 2.3 kb DNA fragment found in the wild-type strain NBRC 0988, i.e., a DNA fragment resulting from amplification of an undisrupted allele, was not detected, this strain was considered a strain in which CuURA3 gene was completely disrupted.

The HygBr strain in which four CuURA3 gene copies were disrupted was transformed with the Cre expression plasmid pCU595. The resulting transformants were plated onto G418 medium and HygB medium, and the results were that they were all able to grow on the G418 medium but were not able to grow on the HygB medium. Using genomic DNA extracted from the HygBs and G418r transformant as a template, PCR was performed with IM-63 (SEQ ID NO:58) and IM-92 (SEQ ID NO:59) (extension reaction: 3.5 minutes). When the resultant products were subjected to 0.8% agarose gel electrophoresis, it was found that there was a plurality of strains from which two DNA fragments, 1.5 kb and 1.1 kb, were amplified. This proved that the HPT gene was eliminated.

After the HygBs strain in which four CuURA3 gene copies were disrupted was cultured overnight in YPD liquid medium, a portion of the culture was plated onto YPD medium. After two days, single colonies were separated and plated onto YPD medium and G418 medium. Then, clones which grew on the YPD medium but did not grow on the G418 medium were separated. A strain in which pCU595 has been omitted, i.e., the HygBs and G418s strain in which fourth CuURA3 gene copy was disrupted was obtained.

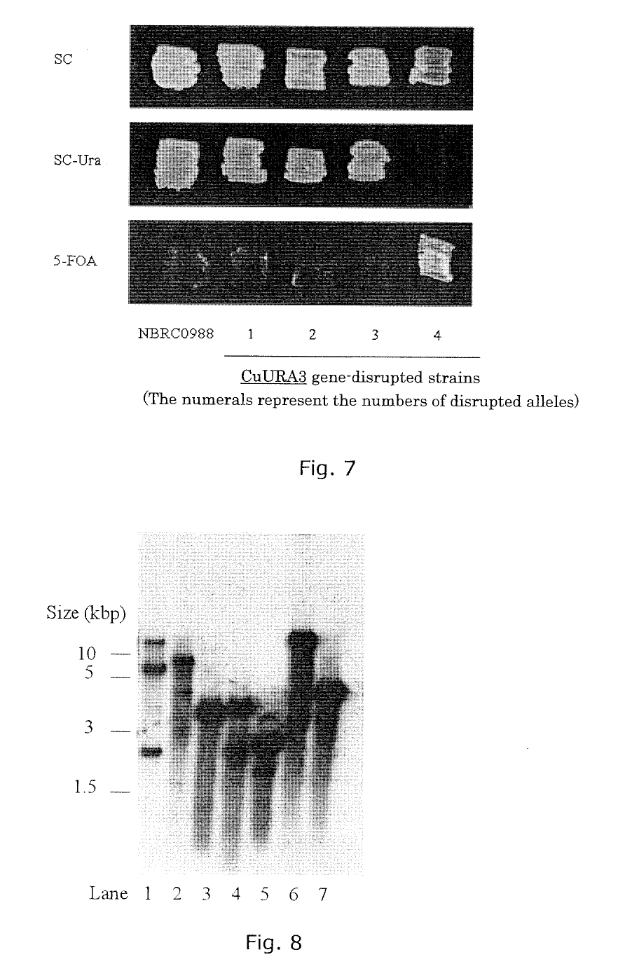

The ability of the NBRC 0988 strain and of the strains obtained by successively disrupting the CuURA3 gene in the NBRC 0988 host strain (which are HygBs and G418s strains in which both the HPT gene and the APT gene have been eliminated) to grow in non-selective SC medium, SC-Ura medium (uracil-free medium), and 5-FOA medium was examined. The compositions of these media followed those described in Methods In Yeast Genetics 1997 Edition (Cold Spring Harbor Laboratory Press). As shown in FIG. 7, only the strain in which four copies of the CuURA3 gene, i.e., all of the CuURA3 genes, have been disrupted was not able to grow in the SC-Ura medium but was able to grow in the 5-FOA medium, unlike the other four strains including the NBRC 0988 strain.

All alleles of the CuURA3 gene were disrupted by means of recombinant DNA techniques, and a uracil auxotrophic strain was successfully obtained. This is the first report demonstrating the existence of four alleles in the cell of Candida utilis. The Candida utilis transformation system utilizing the Cre-loxP system, which was able to efficiently disrupt the genes of interest with repeated use of selectable marker genes, was thought to be of significance.

### Example 2

### Construction of a PDC-Encoding Gene Disrupted Strain

### 2-1. Cloning of the PDC-Encoding Gene

Primers IKSM-29 (SEQ ID NO:1) and IKSM-30 (SEQ ID NO:2) that amplify the base sequence on the side of C-terminus, which contains many common sequences of ScPDC1 gene, KIPDC1 gene etc., were made and subjected to PCR with the genome of the NBRC 0988 strain as a template (extension time: 30 seconds). When the sequence of the amplified DNA fragment of about 220 by (base pairs) (hereinafter referred to as Cup-Fg) was read (SEQ ID NO:3), it was found to be highly homologous to the ScPDC1 gene. This DNA fragment was thus considered part of a PDC-encoding gene.

Using this DNA fragment as a probe, Southern analysis was performed. First, genomic DNA extracted from the Saccharomyces cerevisiae S288C strain (the NBRC 1136 strain) was digested with HindIII, and genomic DNA extracted from Candida utilis NBRC 0988 was digested with XbaI, HindIII, BglII, ExoRI, BamHI, and PstI. These were then subjected to 0.8% agarose gel electrophoresis. The separated genomic DNAs were transferred to a Hybond N⁺ nylon membrane from Amersham Biosciences, according to standard procedures. Random Primer DNA Labelling Kit Ver.2 from TaKaRa was used for radioactive labeling of the probe, and the procedure was carried out according to the attached protocol. As labeled dCTP, 1.85 MBq of [α-³²P]dCTP from Amersham Biosciences was used. Hybridization was performed using Rapid-Hyb buffer, according to the attached protocol with the exception that the temperature of hybridization was 60° C. The results are shown in FIG. 8.

For Saccharomyces cerevisiae, three bands considered to be derived from three PDC genes (the ScPDC1 gene, the ScPDC5 gene, the ScPDC6 gene) were respectively detected (Lane 1). In contrast, for the Candida utilis NBRC 0988 strain, only one band was detected in samples other than those digested with BglII whose restriction enzyme recognition site is present in the probe (Lane4) or with EcoRI (Lanes), suggesting that the number of genes having PDC activity is one in NBRC 0988. Although there is no EcoRI recognition sequence in the probe, it was considered possible that, in the vicinity of the region to which the probe hybridizes on one homologous chromosome, there is an allelic locus that contains an EcoRI recognition sequence, while, on the other homologous chromosome, there is a heterologous region where there is no such sequence.

To make a genomic library utilized for colony hybridization, a reaction for linking a 5 to 10 kb fragment of DNA which was partially digested with Sau3AI to pBR322 (NIPPON GENE) which was digested with BamHI and then dephosphorylated was carried out. With this solution, 50,000 clones, having grown in LB +Amp agar medium, were obtained. Self-ligated clones were less than 5%.

Furthermore, a plurality of clones containing a site of homology to the probe sequence was obtained by colony hybridization using the above DNA fragment CuP-Fg as a probe. Then, the sequences of these clones were read by the primer walking method, and, as a result, one contig was obtained (SEQ ID NO:63). When this was subjected to a BLAST search of SGD (Saccharomyces Genome Database), it was found to be highly homologous to the ScPDC1 gene and to the ScPDC5 gene. For this reason, this gene was designated the CuPDC1 gene. When this DNA fragment was subjected to a homology search with BLAST of NCBI (National Center for Biotechnology Information), it was found to be highly homologous to genes encoding polypeptides having the activity of pyruvate decarboxylase of various yeasts. Further, an attempt to identify an ORF (Open Reading Frame) region was made by conducting a homology search between this sequence and the PDC genes of other species through the use of NCBI database, and the ORF of the CuPDC1 gene was estimated to be 1,692 nt (nucleotides) (SEQ ID NO:63). The sequence of the gene concerned has 76% homology at the amino acid level with the ScPDC1 gene, and it was considered highly possible that it has PDC activity. In addition, it was considered that the 2,246-base sequence of the upstream region of the ORF region of the gene concerned shown in SEQ ID NO:63 corresponds to the promoter region of the CuPDC1 gene, and the 1,076 base sequence of the downstream region corresponds to the terminator region of the CuPDC1 gene. The sequences reported here were all included in pCU530, the plasmid obtained by colony hybridization.

For a 1.2 kb promoter region in the upstream region of the ORF, the existence of cis elements implicated in transcriptional control was examined. The base immediately preceding A of the transcription initiating ATG of the ORF was positioned as “−1.” In consequence, there was a sequence consisting of TATATAA near −149, which was considered a TATA box. At least three sequences (near −1,084, near −998, near −812) highly homologous to the UAS-PDC sequence (GCACCATACCTT) (Butler G., et al., Curr Genet., 14(5):405-12., 1988), which is thought to be necessary for the transcriptional activation of the PDC genes of Saccharomyces cerevisiae and Kluyveromyces lactis, were found. In addition, without overlapping with these sequences, at least four Gcr1-binding domains (near −1,538, near −556, near −518, near −430) and at least five CAAT sequences (near −1,224, near −1,116, near −979, near −658, near −556) were considered to be localized.

Using the DNA fragment Cup-Fg, Southern hybridization was performed on samples prepared by digesting genomic DNAs extracted from Candida utilis strains, such as the NBRC 0626 strain, the NBRC 0639 strain, and the NBRC 1086 strain, with HindIll. In all these samples, bands were detected at the same positions as those detected in NBRC 0988. In addition, in the base sequences of the CuPDC1 gene, single nucleotide polymorphisms were found between the NBRC 0626 strain, the NBRC 0988 strain, and the NBRC 1086 strain.

In order to analyze the function of the CuPDC1 gene, it was examined whether the lethality due to double disruptions of the ScPDC1 gene and the ScPDC5 gene in Saccharomyces cerevisiae could be suppressed by expressing the CuPDC1 gene with the ScPDC1 gene promoter. First, the SGY107 strain, a hybrid strain, was constructed ‘based on a complete series of yeast gene disruption strains’ from the ScPDC1 gene-disrupted strain (Invitrogen), derived from BY4741 (a ura3 gene and his3 gene mutant strain), and the ScPDC5 gene-disrupted strain (Open BioSystems), derived from BY4742 (a ura3 gene and his3 gene mutant strain).

pCU546, a plasmid for expressing the ScPDC1 gene in Saccharomyces cerevisiae, was constructed as follows. Centromeric-type plasmid pRS316 (Sikorski, R. et al., Genetics. 122, 19-27. 1989) (carrying the URA3 gene) that functions in Saccharomyces cerevisiae, was cut with ClaI and BamHI. Using BY4741 (Invitrogen) as a template, PCR was performed with a primer set of IM-135 (SEQ ID NO:4) and IM-136 (SEQ ID NO:5) (extension time: 3 minutes). The amplified fragment was digested with ClaI and BamHI. Plasmid pCU546, consisting of this DNA fragment linked to the plasmid fragment that has previously been treated with the restriction enzymes, was constructed.

Then, the SGY107 strain was transformed with pCU546. This strain was transferred to a sporulation agar medium (0.5 g/L glucose, 1 g/L Yeast Extract, 10 g/L potassium acetate, 20 g/L agarose) and allowed to stand for 3 days at 25° C. Using genomic DNA extracted from the resultant spores as a template, the following two PCRs (extension time: 2 minutes) were performed: (1) a primer set of IM-19 (SEQ ID NO:6) and IM-331 (SEQ ID NO:7) (with this combination, only from a strain in which the ScPDC1 gene has been disrupted, a DNA fragment of about 1.5 kb is amplified); and (2) a primer set of IM-20 (SEQ ID NO:8) and IM-334 (SEQ ID NO:9) (with this combination, only from a strain in which the ScPDC5 gene has been disrupted, a DNA fragment of about 1.5 kb is amplified). The SGY116 strain, from which DNA fragments are amplified with both primer sets and which retains pCU546, was obtained.

A hybrid strain was constructed from the SGY116 strain and the ScPDC6 gene disrupted strain (Open BioSystems) derived from BY4742. This strain was transferred to a sporulation medium and allowed to stand for 3 days at 25° C. Using genomic DNA extracted from the resultant spores was subjected to the following three PCRs (extension time: 2 minutes) were performed: (1) a primer set of IM-19 (SEQ ID NO:6) and IM-331 (SEQ ID NO:7); (2) a primer set of IM-20 (SEQ ID NO:8) and IM-334 (SEQ ID NO:9); and (3) a primer set of IM-339 (SEQ ID NO:10) and IM-340 (SEQ ID NO:11) (with this combination, a DNA fragment amplified from a strain in which the ScPDC6 gene has been disrupted is larger than the DNA fragment (about 3.4 kb) amplified from a strain in which the ScPDC6 gene has not been disrupted). As a result, DNA fragments of about 1.5 kb were amplified by the PCRs in (1) and (2), and a DNA fragment of greater than 3.4 kb was amplified by the PCR in (3). That is, the SGY389 strain in which the ScPDC1 gene, the ScPDC5 gene, and the ScPDC6 gene have all been disrupted and which retains pCU546 was obtained.

pCU655, a plasmid for expressing the CuPDC1 gene in Saccharomyces cerevisiae, was constructed as follows. First, the following PCRs in (1), (2), and (3) were performed: (1) genomic DNA from the BY4741 strain was used as a template, and IM-135 (SEQ ID NO:4) and IM-147 (SEQ ID NO:12) were used as primers (extension reaction: 1 minute); (2) genomic DNA from the BY4741 strain was used as a template, and IM-150 (SEQ ID NO:13) and IM-136 (SEQ ID NO:5) were used as primers (extension reaction: 1 minute); and (3) pCU530 containing the deduced ORF region of the CuPDC1 gene was used as a template, and IM-148 (SEQ ID NO:14) and IM-149 (SEQ ID NO:15) were used as primers (extension reaction: 2 minutes). Then, using the DNA fragments amplified in (1), (2), and (3) as templates, PCR was performed with IM-135 (SEQ ID NO:4) and IM-136 (SEQ ID NO:5) as primers. As a result, a DNA fragment consisting of, in sequence, the ScPDC1 gene, the deduced ORF region of the CuPDC1 gene, and the terminator region of the ScPDC1 gene was obtained. Note that all of these PCRs were performed using KOD-Plus-. This fragment was linked to a DNA fragment obtained by cutting with SmaI the centromeric-type plasmid pRS313 (Sikorski, R. et al.: Genetics. 122, 19-27. 1989) (carrying the HIS3 gene) that functions in Saccharomyces cerevisiae. The resulting plasmid was designated pCU655.

The SGY389 strain, in which the ScPDC1 gene, the ScPDC5 gene, and the ScPDC6 gene have all been disrupted, was transformed with pRS313 or pCU655 to obtain the SGY393 strain and the SGY392 strain, respectively.

In 5-FOA medium, the BY4741 strain, the BY4742 strain, the ScPDC1 gene disrupted strain derived from the BY4741 strain, the ScPDC5 gene disrupted strain derived from the BY4742 strain, and the ScPDC6 gene disrupted strain derived from the BY4742 strain were able to grow. In other words, these strains were proved to be auxotrophic for uracil. Then, the SGY389 strain, in which the ScPDC1 gene, the ScPDC5 gene, and the ScPDC6 gene have all been disrupted and which retains pCU546, and the SGY393 strain, consisting of the SGY389 strain into which pRS313 has been introduced were not able to grow in 5-FOA medium. This proves that pCU546, expressing the ScPDC1 gene, may not be omitted. In other words, it proves that, in 5-FOA medium, expression of a gene encoding a polypeptide having PDC activity is indispensable for growth. On the other hand, the SGY392 strain (a strain that expresses the CuPDC1 gene), consists of the SGY389 strain into which pCU655 has been introduced, was able to grow in 5-FOA medium. This proves that the CuPDC1 gene contained in pCU655 retains the function of PDC that the ScPDC1 gene contained in pCU547 has. Therefore, it was suggested that the polypeptide encoded by the CuPDC1 gene was PDC.

2-2. Multiple Disruptions of the CuPDC1 Genes through the use of the Cre-lox System

A DNA fragment to disrupt the first and second copies of the CuPDC1 gene was prepared as follows. First, the following three PCRs shown in (1), (2), and (3) were performed: (1) pCU563 was used as a template, IM-1 (SEQ ID NO:21) and IM-2 (SEQ ID NO:22) as primers, and the extension reaction time was 2 minutes; (2) genomic DNA from the NBRC 0988 strain was used as a template, IM-277 (SEQ ID NO:27) and IM-278 (SEQ ID NO:28) as primers, and the extension reaction time was 30 seconds; and (3) genomic DNA from the NBRC 0988 strain was used as a template, IM-279 (SEQ ID NO:29) and IM-280 (SEQ ID NO:30) as primers, and the extension reaction time was 30 seconds. In (2) and (3), the upstream portion and downstream portion of the CuPDC1 gene are amplified. Further, the following PCR in (4) was performed: (4) a mixture of the three DNAs amplified in (1), (2), and (3) above was used as a template, IM-277 (SEQ ID NO:27) and IM-280 (SEQ ID NO:30) as primers, and the extension reaction time was 3 minutes. This provided a DNA fragment consisting of, in sequence, the upstream region of the CuPDC1 gene, loxP, the PGK gene promoter, the HPT gene, the GAP gene terminator, loxP, and the downstream region of the CuPDC1 gene. Hereinafter, this DNA fragment is referred to as “the first/second CuPDC1 disruption fragment.” Transformation using this DNA fragment causes double-strand homologous recombination to occur in the upstream region and downstream region of the CuPDC1 gene, thereby making it possible to partially delete an allele of the CuPDC1 gene.

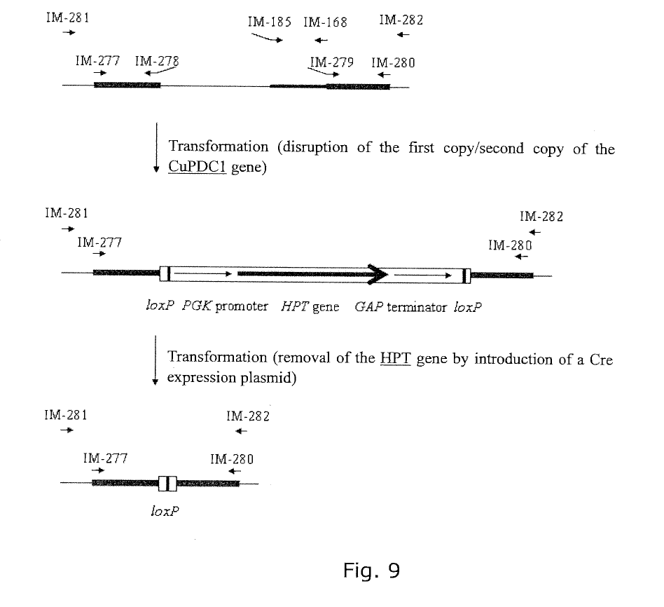

Using the first/second CuPDC1 disruption fragment as a DNA fragment, transformation of the NBRC 0988 strain was performed. Genomic DNAs were extracted from the NBRC 0988 strain and from the resulting transformants and used as templates to perform PCRs with IM-281 (SEQ ID NO:31) and IM-282 (SEQ ID NO:32) (extension reaction: 4 minutes). As illustrated in FIG. 9, these primers anneal outside the homologous recombination area. When the resultant products were subjected to 0.8% agarose gel electrophoresis, it was found that a 3.7 kb DNA fragment was amplified from the NBRC 0988 strain, and 3.9 kb and 3.7 kb DNA fragments were amplified from a plurality of transformants. This proved that a strain of interest of the HygBr in which, one CuPDC1 gene copy was disrupted was obtained.

The HygBr strain in which one CuPDC1 gene copy was disrupted was transformed with the Cre expression plasmid pCU595. In G418-containing medium, the negative control to which DNA was not added did not form a colony, while 1,000 or more transformants were obtained from the sample to which pCU595 was added. Thirty strains randomly selected from them were plated onto G418 medium or HygB medium, and the results were that they were all able to grow on the G418 medium but were not able to grow on the HygB medium.

Genomic DNAs were extracted from the HygBr strain in which one CuPDC1 gene copy was disrupted and from the HygBs strain in which first CuPDC1 gene copy was disrupted, and were used as templates to perform PCRs with IM-281 (SEQ ID NO:31) and IM-282 (SEQ ID NO:32) (extension reaction: 4 minutes). When the resultant products were subjected to 0.8% agarose gel electrophoresis, it was found that 3.9 kb and 3.7 kb DNA fragments were amplified from the former strain, and 3.7 kb and 1.9 kb DNA fragments were amplified from the latter strain. A strain in which the HPT gene has been eliminated as intended was obtained.

After the HygBs strain in which one CuPDC1 gene copy was disrupted was cultured overnight in YPD liquid medium, a portion of the culture was plated onto YPD medium. After two days, a plurality of single colonies were separated and plated onto G418 medium and YPD medium. The results were that most clones grew on the YPD medium but did not grow on the G418 medium.

Genomic DNAs were extracted from the HygBs G418r strain and the HygBs G418s strain, which both have one copy of the CuPDC1 gene disrupted but which are different in the ability to grow in G418-containing medium, and were used as templates to perform PCRs with IM-49 (SEQ ID NO:23) and IM-52 (SEQ ID NO:26), a primer set to amplify the Cre gene (extension reaction: 1 minute). As a result, a 1 kb DNA fragment was amplified from the G418r strain but was not amplified from the G418s strain. Therefore, a HygBs and G418s strain in which first CuPDC1 gene copy was disrupted and pCU595 has been omitted was obtained.

Using the first/second CuPDC1 disruption fragment as a DNA fragment, transformation of the HygBs and G418s strain in which one CuPDC1 gene copy was disrupted was performed. Genomic DNA was extracted from the resulting transformants and used as a template to perform PCR with IM-281 (SEQ ID NO:31) and IM-282 (SEQ ID NO:32) (extension reaction: 4 minutes). When the resultant products were subjected to 0.8% agarose gel electrophoresis, it was found that there was a plurality of strains from which three DNA fragments, 3.9 kb, 3.7 kb, and 1.9 kb, were amplified. This proved that a strain of interest of the HygBr in which two CuPDC1 gene copies were disrupted was obtained.

The HygBr strain in which two CuPDC1 gene copies were disrupted was transformed with the Cre expression plasmid pCU595. When the resulting transformants were plated onto G418 medium and HygB medium, they were all able to grow on the G418 medium but were not able to grow on the HygB medium. Using genomic DNA extracted from the HygBs and G418r transformants as a template, PCR was performed with IM-281 (SEQ ID NO:31) and IM-282 (SEQ ID NO:32) (extension reaction: 4 minutes). When the resultant products were subjected to 0.8% agarose gel electrophoresis, it was found that two DNA fragments, 3.7 kb and 1.9 kb, were amplified. This proved that the HPT gene was eliminated.

After the HygBs strain in which two CuPDC1 gene copies were disrupted was cultured overnight in YPD liquid medium, a portion of the culture was plated onto YPD medium. After two days, single colonies were separated and plated onto YPD medium and G418 medium. Then, clones which grew on the YPD medium but did not grow on the G418 medium were separated. A strain in which pCU595 has been omitted, i.e., the HygBs and G418s strain in which second CuPDC1 gene copy was disrupted was obtained.

A DNA fragment to disrupt the third and forth copies of the CuPDC1 gene was prepared as follows. First, the following three PCRs shown in (1), (2), and (3) were performed: (1) pCU563 was used as a template, IM-1 (SEQ ID NO:21) and IM-2 (SEQ ID NO:22) as primers, and the extension reaction time was 2 minutes; (2) genomic DNA from the NBRC 0988 strain was used as a template, IM-277 (SEQ ID NO:27) and IM-278 (SEQ ID NO:28) as primers, and the extension reaction time was 30 seconds; and (3) genomic DNA from the NBRC 0988 strain was used as a template, IM-185 (SEQ ID NO:33) and IM-168 (SEQ ID NO:34) as primers, and the extension reaction time was 30 seconds. In (2) and (3), the upstream portion and downstream portion of the CuPDC1 gene are amplified. Further, the following PCR in (4) was performed: (4) a mixture of the three DNAs amplified in (1), (2), and (3) above was used as a template, IM-277 (SEQ ID NO:27) and IM-168 (SEQ ID NO:34) as primers, and the extension reaction time was 3 minutes. This provided a DNA fragment consisting of, in sequence, the upstream region of the CuPDC1 gene, loxP, the PGK gene promoter, the HPT gene, the GAP gene terminator, loxP, and the downstream region of the CuPDC1 gene. Hereinafter, this DNA fragment is referred to as “the third/fourth CuPDC1 disruption fragment.” Transformation with this DNA fragment causes double-strand homologous recombination to occur in the upstream region and downstream region of the CuPDC1 gene, thereby making it possible to partially delete an allele of the CuPDC1 gene. In addition, since the downstream region of the CuPDC1 gene amplified in (3) is a region most of which was deleted in the transformations performed using the first/second CuPDC1 disruption fragment to disrupt the first and second copies of the CuPDC1 gene, it is conceivable that the possibility of incorporation into the two disrupted copies of the allele can be largely reduced.

Using the third/fourth CuPDC1 disruption fragment as a DNA fragment, transformation of the HygBs and G418s strain in which two CuPDC1 gene copies were disrupted was performed to disrupt the third copy of the CuPDC1 gene. Genomic DNA was extracted from the resulting transformants and used as a template to perform PCR with IM-281 (SEQ ID NO:31) and IM-282 (SEQ ID NO:32) (extension reaction: 4 minutes). When the resultant products were subjected to 0.8% agarose gel electrophoresis, it was found that there were strains from which three DNA fragments, 4.4 kb, 3.7 kb, and 1.9 kb, were amplified. This proved that a strain of interest of the HygBr in which third CuPDC1 gene copy was disrupted was obtained.

The HygBr strain in which three CuPDC1 gene copies were disrupted was transformed using the Cre expression plasmid pCU595. The resulting transformants were plated onto G418 medium and HygB medium, and the results were that they were all able to grow on the G418 medium but were not able to grow on the HygB medium. Using genomic DNA extracted from the HygBs and G418r transformants as a template, PCR was performed with IM-281 (SEQ ID NO:31) and IM-282 (SEQ ID NO:32) (extension reaction: 4 minutes). When the resultant products were subjected to 0.8% agarose gel electrophoresis, it was found that three DNA fragments, 3.7 kb, 2.4 kb, and 1.9 kb, were amplified. This proved that the HPT gene was eliminated.

After the HygBs strain in which three CuPDC1 gene copies were disrupted was cultured overnight in YPD liquid medium, a portion of the culture was plated onto YPD medium. After two days, single colonies were separated and plated onto YPD medium and G418 medium. Then, clones which grew on the YPD medium but did not grow on the G418 medium were separated. A strain in which pCU595 has been omitted, i.e., the HygBs and G418s strain in which third CuPDC1 gene copy was disrupted was obtained.

Using the third/fourth CuPDC1 disruption fragment as a DNA fragment, transformation of the HygBs and G418s strain in which three CuPDC1 gene copies were disrupted was performed. Genomic DNA was extracted from the resulting transformants and used as a template to perform PCR with IM-281 (SEQ ID NO:31) and IM-282 (SEQ ID NO:32) (extension reaction: 4 minutes). When the resultant products were subjected to 0.8% agarose gel electrophoresis, it was found that there was a plurality of transformants from which three DNA fragments, 4.4 kb, 2.4 kb, and 1.9 kb, were amplified. This proved that a strain of interest the HygBr in which four CuPDC1 gene copies were disrupted was obtained. In addition, since the 3.7 kb DNA fragment found in the wild-type strain NBRC 0988, i.e., a DNA fragment resulting from amplification of an undisrupted allele, was not detected, this strain was considered a strain in which CuPDC1 gene was completely disrupted.

The HygBr strain in which four CuPDC1 gene copies were disrupted was transformed with the Cre expression plasmid pCU595. The resulting transformants were plated onto G418 medium and HygB medium, and the results were that they were all able to grow on the G418 medium but were not able to grow on the HygB medium. Using genomic DNA extracted from the HygBs and G418r transformant as a template, PCR was performed with IM-281 (SEQ ID NO:31) and IM-282 (SEQ ID NO:32) (extension reaction: 4 minutes). When the resultant products were subjected to 0.8% agarose gel electrophoresis, it was found that there was a plurality of strains from which two DNA fragments, 2.4 kb and 1.9 kb, were amplified. This proved that the HPT gene was eliminated.

After the HygBs strain in which four CuPDC1 gene copies were disrupted was cultured overnight in YPD liquid medium, a portion of the culture was plated onto YPD medium. After two days, single colonies were separated and plated onto YPD medium and G418 medium. Then, clones which grew on YPD medium but did not grow on G418 medium were separated. A strain in which pCU595 has been omitted, i.e., the HygBs and G418s strain in which fourth CuPDC1 gene copy was disrupted was obtained. This strain was designated the Cu8402g strain.

### 2-3. Characterization of the CuPDC1 Gene Disrupted Strain

It is considered that the CuPDC1 gene encodes a polypeptide having the activity of pyruvate decarboxylase which catalyzes conversion of pyruvic acid to acetaldehyde. In the fermentation pathway, acetaldehyde is further metabolized to ethanol by alcohol dehydrogenase. That is, disruption of the CuPDC1 gene is expected to shut down the metabolic pathway to ethanol, thereby reducing ethanol production ability. Thus, strains in which one CuPDC1 gene copy was disrupted, two CuPDC1 gene copies were disrupted, three CuPDC1 gene copies were disrupted, and the Cu8402g strain in which CuPDC1 gene was completely disrupted (all of which are HygBs and also G418s) were subjected to fermentation trials, and their ethanol production ability and organic acids were analyzed.

For the purpose of examining the ethanol production ability, aromatic component production ability, and organic acid production ability of the Cu8402g strain, the cells of the wild-type NBRC 0988 strain and the Cu8402g strain, which were grown on YPD agar medium for 1 to 3 days, were inoculated into a new medium (50 mL to 100 mL of YPD liquid medium was used as the medium) to an OD600 of about 0.1, and cultured in a benchtop culture apparatus from TAITEC at an amplitude of 35 mm, at 120 to 150 rpm, at 30° C. for 16 to 30 hours. Then, the cells were collected by centrifugation under the conditions of 4° C., 3,000 rpm, and 5 minutes, and further, after removal of the supernatant, they were washed with the medium (not containing a neutralizing agent) used for fermentation. The two yeast cells thus obtained were each inoculated in 50 mL of YPD10 (100 g/L glucose, 10 g/L yeast extract, 20 g/L peptone) medium contained in a 100 mL baffled Erlenmeyer flask to an initial OD600 of 0.5, and cultured using a benchtop culture apparatus from TAITEC at 30° C. for 48 hours, at an amplitude of 35 mm, and at a shaking speed of 80 rpm. The culture fluid was filtered through a 0.22 μm filter, and the concentrations of ethanol, aromatic components, and various organic acids in the medium were determined. The results are shown in Table 1. The data were calculated from the results of three independent trials.

TABLE 1

| | | | Medium |
|---|---|---|---|
| | NBRC0988 | Cu8402g | (sterile) |
| OD600 | 59.9 ± 2.0 | 2.8 ± 0.2 | N.D.* |
| Glucose | N.D.(G)* | 80 ± 0.6 | 100.2 ± 0.3 |
| (g/L) | | | |
| Ethanol | 3.96 ± 0.04 | N.D.(E)** | N.D.(E)** |
| (g/L) | | | |
| Acetaldehyde | 26.2 ± 9.0 | 1.14 ± 0.06 | 0.30 ± 0.14 |
| (mg/L) | | | |
| Pyruvic acid | 462.4 ± 5.5 | 3659.9 ± 25.6 | N.D.(P)*** |
| (mg/L) | | | |
| Acetic acid | 660.3 ± 41.0 | 273.1 ± 9.3 | 68.6 ± 1.0 |
| (mg/L) | | | |
| L-lactic acid | 78.7 ± 2.9 | 160.0 ± 8.2 | 235.7 ± 4.6 |
| (mg/L) | | | |
| D-lactic acid | 21.8 ± 6.5 | 82.6 ± 9.0 | 52.1 ± 2.8 |
| (mg/L) | | | |
| pH | 4.6 | 3.1 | 6.5 |
| Citric acid | N.D.(O)¥ | N.D.(O)¥ | 28.5 ± 3.0 |
| (mg/L) | | | |
| Malic acid | 18.7 ± 1.2 | N.D.(O)¥ | 63.1 ± 1.0 |
| (mg/L) | | | |
| Succinic acid | 146.6 ± 33.2 | 109.6 ± 0.4 | 90.2 ± 1.8 |
| (mg/L) | | | |

*N.D.(G) indicates that the concentration was less than 0.1 g/L.
**N.D.(E) indicates that the concentration was less than 0.01 g/L.
***N.D.(P) indicates that the concentration was less than 0.1 mg/L.
¥N.D.(O) indicates that the concentration was less than 0.1 mg/L.

Forty-eight hours after the start of fermentation, the production of ethanol was 3.96 g/L in the NBRC 0988 strain, while ethanol was not detectable in the Cu8402g strain.

Forty-eight hours after the start of fermentation, the production of acetaldehyde was 26.2 mg/L in the NBRC 0988 strain, while it was 1.14 mg/L in the Cu8402g strain.

Forty-eight hours after the start of fermentation, the production of acetic acid was 660.3 mg/L in the NBRC 0988 strain, while it was 273.1 mg/L in the Cu8402g strain.

Forty-eight hours after the start of fermentation, the concentrations of ethanol and acetic acid, for which acetaldehyde is a precursor, were both lower in the Cu8402g strain than in the NBRC 0988 strain.

Forty-eight hours after the start of fermentation, the concentration of pyruvic acid in the NBRC 0988 strain was 462.4 mg/L, while 3659.9 mg/L pyruvic acid was observed in the Cu8402g strain. The concentration of L-lactic acid was lower both in the NBRC 0988 strain and in the Cu8402g strain than in the medium to which yeast was not added. The concentration of D-lactic acid was lower in the NBRC 0988 strain but higher in the Cu8402g strain than in the medium to which yeast was not added.

What are characteristic to the Cu8402g strain are the disruption of the CuPDC1 gene, and it is conceivable that the disruptions resulted in shutting down the pathway in which pyruvic acid is converted to acetaldehyde, thereby causing pyruvic acid to accumulate without being metabolized, which in turn facilitated accumulation of D-lactic acid, a precursor of pyruvic acid, in the methylglyoxal pathway.

These results are thought to be attributable to the facts that the CuPDC1 gene encodes pyruvate decarboxylase involved in the conversion of pyruvic acid to acetaldehyde, and that the complete deletion of this gene resulted in eliminating or reducing the activity of the enzyme in the cell.

In the process of purifying L-lactic acid, which is the product of interest, adding calcium carbonate to the culture supernatant and recovering it as L-lactic acid calcium salt is a widely used technique. The considerable reduction in the concentrations of ethanol and of various organic acids of the TCA cycle, compared with the wild-type strain, demonstrates the possibility that the production of by-products or products other than L-lactate in this process can be reduced, and therefore is considered a character that will be a useful indicator for evaluating lactic acid production ability.

Forty-eight hours after the start of fermentation, the strain in which one CuPDC1 gene copy was disrupted two CuPDC1 gene copies were disrupted, and three CuPDC1 gene copies were disrupted had the same level of ethanol production ability as the Candida utilis wild-type strain NBRC 0988.

### Example 3

### Construction of the Candida utilis Strain into which the L-LDH Gene has been Introduced

3-1. Design of the DNA Sequence of the L-LDH Gene which Encodes a Polypeptide having the Activity of L-lactate Dehydrogenase

In order to efficiently express a polypeptide having the activity of L-lactate dehydrogenase derived from bovine, a higher eukaryote, in the yeast Candida utilis, the design and synthesis of a novel gene sequence which does not exist in nature were requested to Takara Bio with the following items as design guidelines with regard to the gene described in Japanese Patent Laid-Open Publication No. 2003-259878 which encodes a polypeptide having the activity of lactate dehydrogenase as set forth in a bovine-derived enzyme's amino acid sequence (DDBJ/EMBL/GenBank Accession number: AAI46211.1).
(A) Codons that are frequently used in Candida utilis were used.
(B) mRNA instability sequences and repeated sequences were eliminated as much as possible.
(C) Variation of GC content was adjusted not to differ throughout the entire region.
(D) Unsuitable restriction enzyme sites for gene cloning were prevented from being included in the designed sequence.
(E) Useful restriction enzyme sites were added to the ends for incorporation into an L-LDH gene expression vector (upstream of the L-LDH coding region: KpnI, XbaI; downstream of the L-LDH coding region: BamHI, SacI). Here, the KpnI recognition site refers to the sequence GGTACC from g at position 1 to c at position 6 in the nucleotide sequence of SEQ ID NO:36; the Xba I recognition site refers to the sequence TCTAGA from t at position 7 to a at position 12 in the nucleotide sequence of SEQ ID NO:36; the BamHI recognition site refers to the sequence GGATCC from g at position 1,015 to c at position 1,020 in the nucleotide sequence of SEQ ID NO:36; and the Sad recognition site refers to the sequence GAGCTC from g at position 1,021 to c at position 1,026 in the nucleotide sequence of SEQ ID NO:36.

The synthesized DNA sequence is shown in SEQ ID NO:36. The nucleotide sequence from a at position 13 to a at position 1,011 of SEQ ID NO:36 encodes the polypeptide having the activity of L-lactate dehydrogenase described above, and the amino acid sequence corresponding to this nucleotide sequence is bovine-derived per se and shown as SEQ ID NO:35 (DDBJ/EMBL/GenBank Accession number: AAI46211.1). TGA at positions 1,009 to 1011 and subsequent TGA at positions 1,012 to 1,014 of SEQ ID NO:36 are both translation termination codons. The plasmid containing this DNA fragment was designated pCU669 (another name: GA07033).

FIG. 1 depicts an alignment of the nucleotide sequence (codon optimized sequence) from a at position 13 to a at position 1,011 (the upstream TGA of the two translation termination codons) of SEQ ID NO:36 and the nucleotide sequence shown in SEQ ID NO:38 (wild-type sequence from bovine). These sequences share 751 out of 999 bases, with a homology of 75%. In FIG. 1, the upper sequence is the nucleotide sequence from a at position 13 to a at position 1,011 (the upstream TGA of the two translation termination codons) of SEQ ID NO:36. The lower sequence in FIG. 1 is the base sequence of the L-LDH-A gene derived from Bos taurus (taken from DDBJ/EMBL/GenBank Accession number: BC146210.1) shown in SEQ ID NO:38 (whose translational product is shown in SEQ ID NO:35).

### 3-2. Preparation of a Plasmid for Expressing the L-LDH Gene

Construction of a plasmid for expressing the L-LDH gene was carried out as follows using KOD-Plus-, unless otherwise specified.

PCR was performed with IM-345 (SEQ ID NO:39) and IM-346 (SEQ ID NO:40) to amplify the downstream region of the CuPDC1 gene (extension reaction: 1 minute). After digested with BssHII, the amplified fragment was linked to pBluescriptIISK(+) (TOYOBO) digested completely with BssHII. The resulting plasmid was designated pCU670 (another name: pPt).

pCU621, a plasmid carrying a PGK gene promoter made longer than the one carried by the plasmid pCU563 to prepare a DNA fragment for gene disruption, was constructed according to the following procedure. Using as a template the plasmid pGKHPT1 described in Shimada et al. (Appl. Environ. Microbiol. 64, 2676-2680), which carries the PGK gene promoter and the HPT gene (hygromycin-resistant gene), PCR (extension reaction: 2 minutes) was performed with a primer set of IM-283 (SEQ ID NO:41) and IM-57 (SEQ ID NO:17) to amplify a DNA fragment that consists of, in sequence, loxP, the PGK gene promoter, and the HPT gene.

In addition, using pGAPPT10 (Kondo et al., Nat. Biotechnol. 15, 453-457) as a template, PCR (extension reaction: 30 seconds) was performed with a primer set of IM-54 (SEQ ID NO: 19) and IM-55 (SEQ ID NO:20) to amplify a DNA fragment consisting of the GAP gene terminator and loxP. These were mixed together and subjected to PCR with IM-1 (SEQ ID NO:21) and IM-2 (SEQ ID NO:22) (extension reaction: 2.5 minutes, the enzyme used: LA Taq from Takara Bio) to amplify a DNA fragment consisting of, in sequence, loxP, the PGK gene promoter, the HPT gene, the GAP gene terminator, and loxP. The resulting DNA fragment was cloned into a pCR2.1 vector. The plasmid thus obtained was designated pCU621 (another name: pNNLHL).

First, the following three PCRs were performed: (1) PCR with pCU621 as a template and IM-349 (SEQ ID NO:42) and IM-350 (SEQ ID NO:43) as primers was performed (extension reaction: 2.5 minutes); (2) PCR with pPGKPT2 (Japanese Patent Application Laid-Open No. 2003-144185) as a template and IM-347 (SEQ ID NO:44) and IM-348 (SEQ ID NO:45) as primers was performed (extension reaction: 30 seconds) to amplify the PGK gene terminator region with a single nucleotide mutation introduced such that the terminator region no longer had the BglII recognition sequence; and (3) PCR with the DNA fragments amplified in (1) and (2) as templates and IM-347 (SEQ ID NO:44) and IM-350 (SEQ ID NO:43) as primers was performed (extension reaction: 3 minutes). The DNA fragment obtained in (3) was linked to pBluescriptIISK(+) digested with SmaI. The resulting plasmid was designated pCU672 (another name: pPGtH).

A DNA fragment of about 3 kbp consisting of the PGK gene terminator, loxP, the PGK gene promoter, the HPT gene, the GAP gene terminator, and loxP, obtained by digesting pCU672 (another name: pPGtH) with BamHI and Clal, was linked to pCU670 digested with BamHI and Clal to construct a new plasmid pCU675 (another name: pPGtHPt).

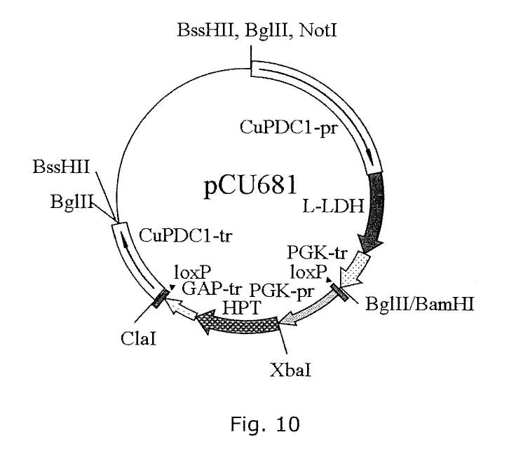

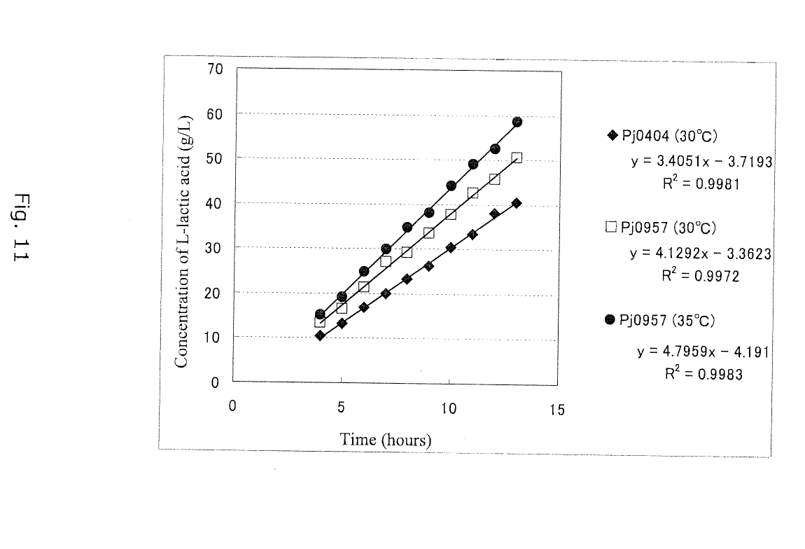

First, the following three PCRs were performed: (1) PCR with pCU530 as a template and IM-341 (SEQ ID NO:46) and IM-342 (SEQ ID NO:47) as primers was performed (extension reaction: 2 minutes) to amplify the CuPDC1 gene promoter region; (2) PCR with pCU669 (another name: GA07033) as a template and IM-343 (SEQ ID NO:48) and IM-379 (SEQ ID NO:49) as primers was performed (extension reaction: 1 minute) to amplify the L-LDH structural gene; and (3) PCR with the DNA fragments amplified in (1) and (2) as templates and IM-341 (SEQ ID NO:46) and IM-379 (SEQ ID NO:49) as primers was performed (extension reaction: 3 minutes). The DNA fragment amplified in (3) was digested with NotI and BglII, and the resulting DNA fragment was linked to pCU675 (another name: pPGtHPt) cut with NotI and BamHI. In the resulting plasmid pCU681 (another name: pPLPGtHPt) (FIG. 10), a DNA fragment consisting of, in sequence, the CuPDC1 gene promoter region, the L-LDH structural gene, the PGK gene terminator, loxP, the PGK gene promoter, the HPT gene, the GAP gene terminator, loxP, and the downstream region of the CuPDC1 gene has been inserted into the BssHII sites of pBluescriptIISK(+). On the inserted DNA fragment side of each BssHII recognition sequence, there is a BglII recognition sequence immediately behind the recognition sequence. Thus, the aforementioned DNA fragment consisting of from the CuPDC1 gene promoter to the downstream region of the CuPDC1 gene can be obtained by digesting pCU681 (another name: pPLPGtHPt) with BglII. Transformants are selected on YPD medium containing 600 _{f}ag/mL hygromycin B.

3-3. Introduction of the L-LDH Gene into the Candida utilis Wild-Type Strain NBRC 0988

The NBRC 0988 strain was transformed with 3 pg of pCU681 (pPLPGtHPt) digested with BglII. Using DNA extracted from the resulting transformants as a template, PCR was performed with a primer set of IM-362 (SEQ ID NO:50) and IM-174 (SEQ ID NO:51) (extension reaction: 4 minutes). From the transformants, the transformant Pj0202 strain, from which a 3.6 kb DNA fragment that is not amplified from the NBRC 0988 strain is amplified, was obtained. In addition, when PCR with a primer set of IM-163 (SEQ ID NO:52) and IM-164 (SEQ ID NO:53) was performed (extension reaction: 30 seconds), a DNA fragment of about 500 by was amplified. This proved that the Pj0202 strain has at least one or more undisrupted copies of the CuPDC1 gene.

3-4. Introduction of the L-LDH Gene into the NBRC 0988 Strain and into the Cu8402g Strain in which the CUPDC1 Gene is Completely Disrupted

The Cu8402g strain was transformed with 3 μg of pCU681 (pPLPGtHPt) digested with BglII. Using DNA extracted from the resulting HygBr transformants as a template, PCR was performed with a primer set of IM-362 (SEQ ID NO:50) and IM-174 (SEQ ID NO:51) (extension reaction: 4 minutes). From the transformants, the transformant Pj0404 strain, from which a 3.6 kb DNA fragment that is not amplified from the Cu8402g strain is amplified, was obtained. This strain is a strain in which the L-LDH gene has been incorporated into the CuPDC1 locus, and the expression of the L-LDH gene is controlled by the original CuPDC1 gene promoter.

The Pj0404 strain is a strain into which at least one or more copies of the L-LDH gene have been introduced.

Particularly, since selection of a transformant showing a phenotype of HygBr is made possible by introducing only one copy of the HPT gene, which was expressed in this study, it is conceivable that the Pj0404 strain is a strain into which one copy of the L-LDH gene has been incorporated. PCR was performed with a primer set of IM-281 (SEQ ID NO:31) and IM-282 (SEQ ID NO:32) (extension reaction 4 minutes), and the results were that at least two DNA fragments, 2.4 kb and 1.9 kb, were amplified. The results revealed that the Pj0404 strain contains a disrupted CuPDC1 locus into which the L-LDH gene has not been incorporated.

The Pj0404 strain was transformed with the Cre recombinase expression plasmid pCU595 to obtain a HygBs and G418r clone. After the clone was cultured overnight in YPD liquid medium, a portion of the culture was plated onto YPD medium. After two days, single colonies were separated and plated onto YPD medium and G418 medium. Then, clones which grew on the YPD medium but did not grow on the G418 medium were separated. Using DNA extracted from this clone Pj0707a strain as a template, PCR was performed with a primer set of IM-362 (SEQ ID NO:50) and IM-174 (SEQ ID NO:51) (extension reaction: 4 minutes), and the results were that 1.2 kb DNA was amplified.

The Pj0707a strain is a strain having a phenotype of HygBs and G418s, in which the CuPDC1 genes have all been disrupted, and into which the L-LDH gene driven by the CuPDC1 gene promoter incorporated into the CuPDC1 locus has been introduced.

The Pj0707a strain was transformed with 3 pg of pCU681 (pPLPGtHPt) digested with BglII. Using DNA extracted from the resulting HygBr transformant as a template, PCR was performed with a primer set of IM-362 (SEQ ID NO:50) and IM-174 (SEQ ID NO:51) (extension reaction: 4 minutes). As a result, the transformant Pj0957 strain, from which two DNA fragments, 3.6 kb and 1.2 kb, are amplified, was obtained. This strain is a strain in which the L-LDH gene has been incorporated into the CuPDC1 locus of an allele different from the CuPDC1 locus into which the L-LDH gene has been incorporated in the Pj0457 strain. The expression of the L-LDH gene introduced by this transformation is also controlled by the original CuPDC1 gene promoter.

The Pj0957 strain is a strain into which at least two or more copies of the L-LDH gene have been introduced.

Particularly, since selection of a transformant showing a phenotype of HygBr is made possible by introducing only one copy of the HPT gene, which was expressed in this study, it is conceivable that the Pj0957 strain is a strain into which two copies of the L-LDH gene have been incorporated.

It was confirmed that the Cre-loxP system in Candida utilis can be utilized not only to disrupt a gene, but also to introduce any gene.

### Example 4

### Fermentation Trials in a Flask

As shown below, evaluation of the lactic acid production ability of the NBRC 0988 strain and of newly constructed recombinant yeast strains was performed. The concentration of ethanol in medium was determined by using GC or HPLC, and the concentrations of glucose and L-lactic acid in a medium were determined by using Biochemistry Analyzer (BA) from YSI JAPAN. An F-kit D-lactic acid/L-lactic acid from J.K. International was used to distinguish between optical isomers, and the procedure was carried out according to the attached protocol. The amounts of the production of other various organic acids were determined by using HPLC. Before used as a sample subjected to the analysis, the culture fluid was filtered through a 0.22 μm filter. The data are the mean values of the results of at least three independent trials.

A loopful of a strain in pellet-like form, scraped using a platinum loop from yeast cells grown on YPD agar medium for 2 to 3 days at 30° C., was inoculated into 3 mL of YPD liquid medium contained in a 15 mL tube and pre-precultured for 20 to 30 hours at 30° C. in a benchtop culture apparatus from TAITEC at an amplitude of 35 mm at 130 rpm. This was inoculated into 100 mL of YPD medium contained in a Sakaguchi flask to an OD600 of about 0.1, and was precultured at 30° C. in a benchtop culture apparatus from TAITEC at an amplitude of 35 mm at 130 rpm, typically for 15 to 22 hours. Then, the cells were collected by centrifugation under the conditions of 4° C., 3,000 rpm, and 5 minutes, and further, after removal of the supernatant, they were washed with the medium (not containing a neutralizing agent) used for fermentation. The cells thus obtained were inoculated into a 15 mL volume of a medium containing 100 to 115 g/L glucose in a 100 mL baffled Erlenmeyer flask, and were fermented in a benchtop culture apparatus from TAITEC at an amplitude of 35 mm at 80 rpm. With regard to the amount of the cells inoculated for fermentation, the cells derived from the preculture were inoculated to an OD600 of 10, unless otherwise stated. Unless otherwise specified, calcium carbonate was added to the medium as a neutralizing agent to a concentration of 4.5% (w/v). The temperature during the fermentation was set at 25° C., 30° C., or 35° C. Besides the above concentration (100 to 115 g/L) of glucose, 10 g/L yeast extract and 20 g/L peptone were added to the medium. The medium of this composition is hereinafter referred to as YPD10 medium.

The total sugar conversion rate (%) is a value obtained by dividing the weight of L-lactic acid in a medium by an initial weight of glucose in the medium and then multiplying it by 100. The optical purity (%) of L-lactic acid is a value obtained by dividing the L-lactic acid concentration by the L-lactic acid concentration plus the D-lactic acid concentration and then multiplying it by 100.

For the NBRC 0988 strain, the Cu8402g strain, and the Pj0202 strain, the concentrations of glucose, ethanol, L-lactic acid, D-lactic acid, and other organic acids in the media 24 hours after the start of fermentation were determined. The results at the fermentation temperature of 30° C. are listed in Table 2.

TABLE 2

| | NBRC0988 | Cu8402g | Pj0202 |
|---|---|---|---|
| Initial glucose | 104.3 ± 2.6 | 106.3 ± 1.2 | 110.5 ± 0.4 |
| (g/L) | | | |
| Glucose | N.D.(G)* | 74.1 ± 5.6 | N.D.(G)* |
| (g/L) | | | |
| Ethanol | 36.4 ± 0.7 | N.D.(E)** | 13.7 ± 0.7 |
| (g/L) | | | |
| Pyruvic acid | 1.60 ± 0.10 | 23.1 ± 3.4 | 0.673 ± 0.043 |
| (g/L) | | | |
| L-lactic acid | N.D.(LL)*** | N.D.(LL)*** | 69.0 ± 0.9 |
| (g/L) | | | |
| D-lactic acid | 0.184 ± 0.020 | 0.724 ± 0.030 | 0.0907 ± 0.0082 |
| (g/L) | | | |
| Total sugar | N.D.# | N.D.# | 62.4 ± 1.1 |
| conversion rate | | | |
| (%) | | | |
| pH | 6.7 | 5.4 | 6.0 |
| Citric acid | 190.0 ± 10.9 | 120.5 ± 23.1 | 74.8 ± 7.0 |
| (mg/L) | | | |
| Malic acid | 109.8 ± 11.4 | N.D.(O)**** | N.D.(O)**** |
| (mg/L) | | | |
| Succinic acid | 488.9 ± 26.7 | 251.4 ± 5.9 | 190.0 ± 29.0 |
| (mg/L) | | | |

*N.D.(G) indicates that the concentration was less than 0.1 g/L.
**N.D.(E) indicates that the concentration was less than 0.01 g/L.
***N.D.(LL) indicates that the concentration was less than 0.1 g/L.
****N.D.(O) indicates that the concentration was less than 0.1 mg/L.
#N.D. indicates that the conversion rate was less than 0.1%.

These results proved that, compared with the NBRC 0988 strain, the Cu8402g strain in which the CuPDC1 genes have been completely disrupted produces little or no L-lactic acid and ethanol, and accumulates pyruvic acid and D-lactic acid in high quantities.

According to the results in Table 2, the wild-type strain NBRC 0988 consumed almost all the glucose and produced ethanol. In addition, the concentration of L-lactic acid in the NBRC 0988 strain was lower. The Pj0202 strain, which carries both an undisrupted CuPDC1 gene and the L-LDH gene, produced both ethanol and L-lactic acid. It was considered that such means as reducing the amount of ethanol production, e.g., deleting all the CuPDC1 genes, is effective to enhance the efficiency of L-lactic acid production from glucose.

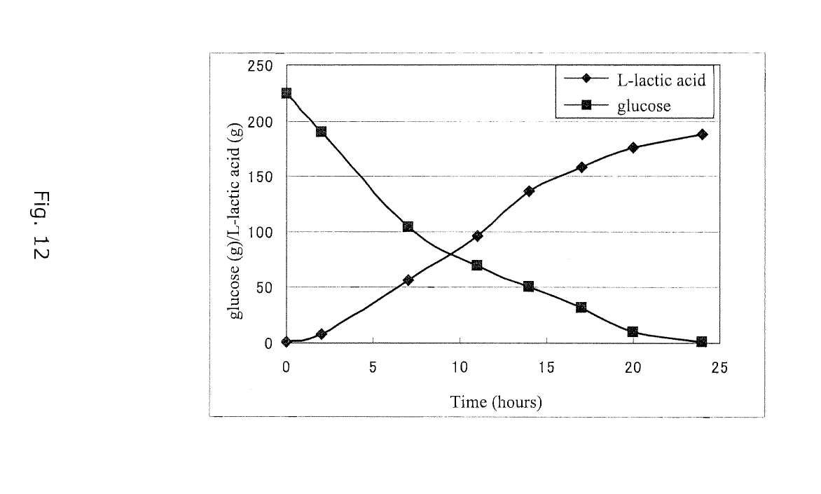

For the Pj0404 strain and the Pj0957 strain, the concentrations of L-lactic acid in the media were determined hourly from 4 to 13 hours after the start of fermentation. With regard to the fermentation temperature, the one condition of 30° C. was used for the Pj0404 strain, and the two conditions of 30° C. and 35° C. were used for the Pj0957 strain. For the respective data sets, linear approximate expressions were also determined. The results are illustrated in FIG. 11.

The rates of L-lactic acid production per unit time were 3.41 g/L/hour (r-squared value =0.998) for the Pj0404 strain (30° C.), 4.13 g/L/hour (r-squared value =0.997) for the Pj0957 strain (30° C.), and 4.80 g/L/hour (r-squared value =0.998) for the Pj0957 strain (35° C.). These results proved that the Pj0957 strain, which carries the larger number of copies of the L-LDH gene than the Pj0404 strain, has the ability to produce lactic acid faster. In addition, to increase the fermentation rate of the Pj0957 strain, it was considered better to select 35° C. than 30° C.

For the Pj0404 strain and the Pj0957 strain, the concentrations of glucose, ethanol, L-lactic acid, D-lactic acid, and other organic acids in the media were determined 24 hours after the start of fermentation. With regard to the fermentation temperature, the one condition of 30° C. was used for the Pj0404 strain, and the two conditions of 30° C. and 35° C. were used for the Pj0957 strain. The results are listed in Table 3.

TABLE 3

| | Pj0404 | Pj0957 | Pj0957 |
|---|---|---|---|
| | (30° C.) | (30° C.) | (35° C.) |
| Initial glucose | 102.7 ± 2.4 | 107.7 ± 1.7 | 108.7 ± 0.5 |
| (g/L) | | | |
| Glucose | 28.0 ± 1.5 | 21.3 ± 1.6 | 10.1 ± 0.9 |
| (g/L) | | | |
| Ethanol | N.D.(E)* | N.D.(E)* | N.D.(E)* |
| (g/L) | | | |
| Pyruvic acid | 0.839 ± 0.036 | 0.199 ± 0.029 | 0.180 ± 0.038 |
| (g/L) | | | |
| L-lactic acid | 72.5 ± 1.7 | 78.1 ± 1.3 | 93.5 ± 0.6 |
| (g/L) | | | |
| D-lactic acid | 0.0277 ± 0.001 | 0.0221 ± 0.003 | 0.0269 ± 0.003 |
| (g/L) | | | |
| Optical purity | 99.92 ± 0.005 | 99.94 ± 0.006 | 99.94 ± 0.007 |
| (%)** | | | |
| Total sugar | 70.65 ± 0.08 | 72.52 ± 1.61 | 86.10 ± 0.51 |
| conversion rate | | | |
| (%) | | | |
| Citric acid | 58.0 ± 3.0 | 55.3 ± 0.5 | N.D.(O)**** |
| (mg/L) | | | |
| Malic acid | N.D.(O)**** | N.D.(O)**** | N.D.(O)**** |
| (mg/L) | | | |
| Succinic acid | 245.3 ± 18.7 | 171.5 ± 21.8 | 129.4 ± 0.9 |
| (mg/L) | | | |

*N.D.(E) indicates that the concentration was less than 0.01 g/L.
**The optical purity is shown as the average value of individual data and their standard deviation.
****N.D.(O) indicates that the concentration was less than 0.1 mg/L.

Twenty-four hours after the start of fermentation, 4.5% (w/v) calcium carbonate added as a neutralizing agent remained in powder form in all the samples. In addition, the amount of lactic acid production of the Pj0404 strain was lower than that of the Pj0957 strain. This proved that the larger the number of copies of the L-LDH gene is, the faster the lactic acid production is.

Twenty-four hours after the start of fermentation, the amount of lactic acid produced by culturing the Pj0957 strain at 35° C. was larger than that produced by culturing the Pj0957 strain at 30° C. It was therefore considered that in terms of the fermentation temperature, 35° C. is preferred for the production of L-lactic acid over 30° C.

As for the Pj0957 strain, fermentations were performed with adding a neutralizing agent and without adding it, respectively. The fermentation temperature was set at 35° C. Table 4 lists the results of the determination of the concentrations of glucose, ethanol, L-lactic acid, D-lactic acid, and other organic acids in the media after 33 hours.

TABLE 4

| | Pj0957 | Pj0957 |
|---|---|---|
| | (with a | (without a |
| | neutralizing agent) | neutralizing agent) |
| Initial glucose(g/L) | 108.7 ± 0.5 | 106.7 ± 0.5 |
| Glucose(g/L) | N.D.(G)* | 65.5 ± 2.4 |
| Ethanol(g/L) | N.D.(E)** | N.D.(E)** |
| Pyruvic acid(g/L) | 0.291 ± 0.020 | 0.014 ± 0.008 |
| L-lactic acid(g/L) | 103.3 ± 0.94 | 38.4 ± 1.7 |
| D-lactic acid(g/L) | 0.0214 ± 0.004 | 0.0140 ± 0.003 |
| pH | 4.0 | 2.9 |
| Optical purity(%)*** | 99.96 ± 0.008 | 99.92 ± 0.017 |
| Total sugar | 95.10 ± 1.13 | 36.03 ± 1.64 |
| conversion rate(%) | | |
| Citric acid(mg/L) | 32.9 ± 1.9 | 58.4 ± 0.3 |
| Malic acid(mg/L) | N.D.(O)**** | N.D.(O)**** |
| Succinic acid(mg/L) | 133.7 ± 2.6 | 158.4 ± 9.7 |

*N.D.(G) indicates that the concentration was less than 0.1 g/L.
**N.D.(E) indicates that the concentration was less than 0.01 g/L.
***The optical purity is shown as the average value of individual data and their standard deviation.
****N.D.(O) indicates that the concentration was less than 0.1 mg/L.

In the case of adding no neutralizing agent, the amount of lactic acid production was less than that in the case of adding a neutralizing agent. It is conceivable that this is caused by the intense acidity of the medium. It was therefore considered that addition of a neutralizing agent is effective in efficiently producing lactic acid.

The Pj0957 strain in which the CuPDC1 genes have been completely disrupted, and further, into which the L-LDH genes have been introduced produced L-lactic acid from a medium containing 108.7 g/L glucose with a high efficiency of total sugar conversion rate of 95.10% within a short period of time.

For the Pj0957 strain, the L-lactic acid concentrations in the YPD10 medium (containing 100 g/L glucose) to which 4.5% (w/v) of calcium carbonate has been added were determined every 2 hours from 4 to 12 hours after the start of fermentation with an initial OD of 10 and a fermentation temperature of 25° C. A linear approximate expression for the concentrations was determined, and the rate of L-lactic acid production per unit time was calculated to be 3.0 g/L/h. Furthermore, when the concentration of L-lactic acid at 33 hours after the start of fermentation was determined, the L-lactic acid concentration in the medium was 95 g/L. Although the rate of L-lactic acid production was not as high as those under the conditions of 30° C. and 35° C., a comparable amount of L-lactic acid was produced under the condition of 25° C. Thus, it was revealed that the Pj0957 strain can produce L-lactic acid with high efficiencies over a wide range of temperatures from 25° C. to 35° C.

The amount of cells to be subjected to fermentation was investigated. The Pj0404 strain and the Pj0957 strain were inoculated such that the starting OD600 of fermentation was 2, 5 or 10, and the concentrations of glucose and L-lactic acid in the media at 42.5 hours after the start of fermentation were determined. A medium with a sugar concentration of 100 g/L was used. The volume of the medium was 15 mL.

For the Pj0404 strain, the results were 88.2 g/L under the condition of OD of 2, 92.0 g/L under the condition of OD of 5, and 93.0 g/L under the condition of OD of 10. For the Pj0957 strain, the results were 93.8 g/L under the condition of OD of 2, 92.2 g/L under the condition of OD of 5, and 92.8 g/L under the condition of OD of 10. These results proved that even when the initial OD is lower than 10, production of L-lactic acid with almost the same efficiency as under the condition of OD of 10 is made possible by increasing the fermentation time.

### Example 5

### Fermentation Trials using a Jar Fermenter

As shown below, evaluation of the lactic acid production ability of the Pj0957 strain was performed. The concentration of ethanol in medium was determined by using GC or HPLC, and the concentrations of glucose and L-lactic acid in the medium were determined by using Biochemistry Analyzer (BA) from YSI JAPAN.

An F-kit D-lactic acid/L-lactic acid from J.K. International was used to distinguish between optical isomers, and the procedure was carried out according to the attached protocol. The amounts of the production of other various organic acids were determined by using HPLC. Before used as a sample subjected to the analysis, the culture fluid was filtered through a 0.22 μm filter.

A loopful of a strain in pellet-like form, scraped using a platinum loop from yeast cells grown on YPD agar medium for 2 to 3 days at 30° C., was inoculated into 3 mL of the YPD liquid medium contained in a 15 mL tube and pre-pre-precultured for 6 to 15 hours at 30° C. in a benchtop culture apparatus from TAITEC at an amplitude of 35 mm at 130 rpm. Subsequently, as pre-preculture, the cells from the pre-pre-preculture were inoculated into a new medium (50 mL of YPD liquid medium was used as the medium) to an OD600 of about 0.1, and then grown at 30° C. at 130 rpm, typically for 12 to 18 hours, to a logarithmic growth phase or stationary phase, indicated by an OD600 of 10 to 25. In this culture, a Sakaguchi flask was used. With regard to the conditions employed in the preculture for preparing cells to be subjected to fermentation, the total amount of cells from the pre-preculture were inoculated into 2.5 L of YPD medium in a 5L-volume jar fermenter (the benchtop culture apparatus Bioneer-C 5L (S), from B.E. MARUBISHI), and grown for 21 to 27 hours at 400 rpm, 30° C., and 1 vvm. During this period, the culture generally exhibited an OD600 of 10 to 25. Then, the cells were collected by centrifugation under the conditions of 4° C., 3,000 rpm, and 5 minutes, and further, after removal of the supernatant, they were washed with the medium (not containing a neutralizing agent) used for fermentation. The cells thus obtained were inoculated into a 2 L volume of the medium containing 100 to 120 g/L glucose, and fermented in a 5 L-volume jar fermenter (the benchtop culture apparatus Bioneer-C 5L (S), from B.E. MARUBISHI). With regard to the amount of the cells inoculated for fermentation, the cells derived from the preculture were inoculated to an OD600 of 10. In this fermentation trial, the Pj0957 strain was used. The agitation speed was set at 250 rpm, the temperature at 35° C., and the aeration rate at 1 vvm. As neutralization conditions, the case of adding calcium carbonate to the medium to a concentration of 5% (w/v) at the start of fermentation and the case of setting a program to perform feedback control such that the pH during fermentation is controlled at 5.5 with 2.5 N sodium hydroxide were investigated. The results with calcium carbonate are values obtained from only one trial, and the results with sodium hydroxide are the average of values independently obtained from two trials. In addition, since the volume of the fermented solution largely changes in the case of using sodium hydroxide, the volume of the medium was determined from the amount of sodium hydroxide added. In this investigation, the value obtained from analysis by HPLC, BA, etc., being a concentration, was multiplied by the volume of the medium, i.e., the total weight of the substance was determined.

The total sugar conversion rate (%) is a value obtained by dividing the weight of L-lactic acid in the medium by an initial weight of glucose in the medium and then multiplying it by 100. The optical purity (%) of L-lactic acid is a value obtained by dividing L-lactic acid concentration by L-lactic acid concentration plus D-lactic acid concentration and then multiplying it by 100.

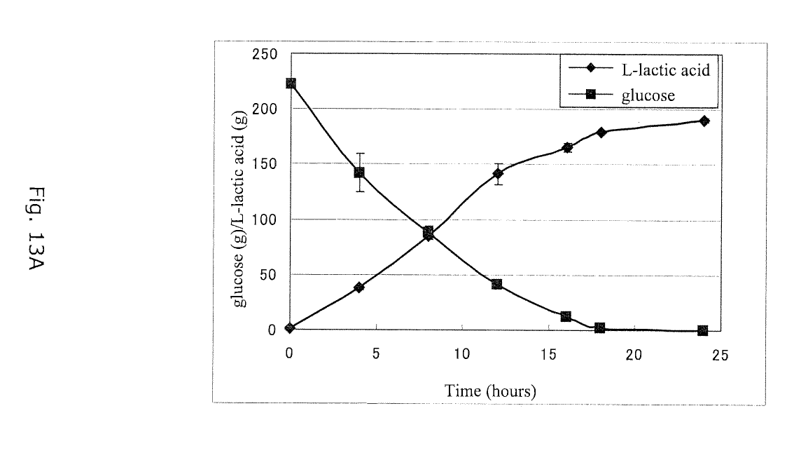

In the trial using calcium carbonate as a neutralizing agent, sampling was performed multiple times within 24 hours after the start of fermentation. FIG. 12 illustrates a graphical representation showing changes in the amounts of glucose and L-lactic acid in the medium over time.

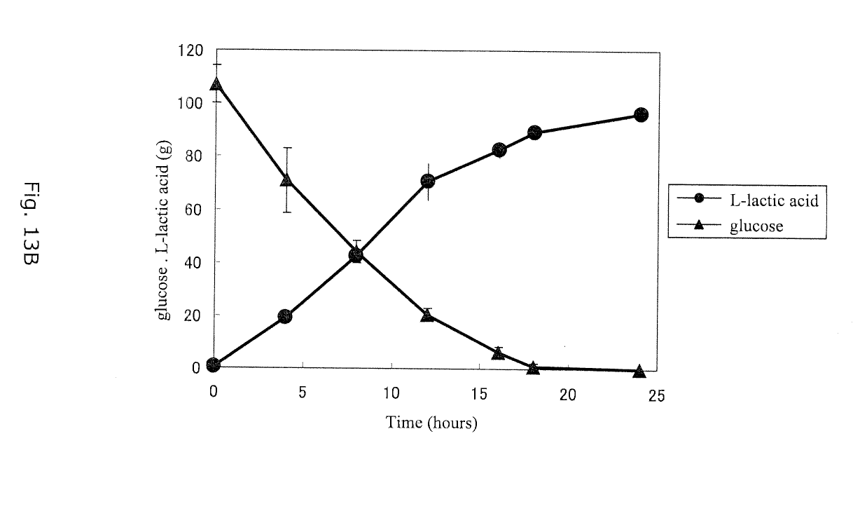

In the trial using sodium hydroxide as a neutralizing agent, sampling was performed multiple times within 24 hours after the start of fermentation. FIG. 13A illustrates a graphical representation showing changes in the amounts of glucose and L-lactic acid in the medium over time (n=2).

The amounts of glucose, ethanol, L-lactic acid, D-lactic acid, and other organic acids as well as pH in the media at 24 hour after the start of fermentation were examined. The results are listed in Table 5 (n=2). After 24 hours, no powders (solid matter) of the added calcium carbonate were observed.

TABLE 5

| | When calcium | When sodium |
|---|---|---|
| | carbonate was used | hydroxide was used |
| Initial glucose(g/L) | 224 | 222.0 ± 4.0 |
| Initial medium | 2 | 2 |
| volume(L) | | |
| Medium volume(L) | 2 | 2.89 ± 0.03 |
| Glucose(g) | 1.4 | less than 0.1 g |
| Ethanol(g) | less than 0.1 g | less than 0.1 g |
| Pyruvic acid(mg) | 69.1 | 69.7 ± 2.9 |
| L-lactic acid(g) | 188 | 190.3 ± 2.1 |
| D-lactic acid(g) | 0.085 | less than 0.01 g |
| Total sugar conversion | 83.9 | 85.7 ± 0.6 |
| rate(%) | | |
| Optical purity | 99.9% or higher | 99.9% or higher |
| pH | 4.4 | 5.5 |
| Citric acid(mg) | less than 20 mg | less than 20 mg |
| Malic acid(mg) | 16.3 | 21.2 ± 0.1 |
| Succinic acid(mg) | 46.1 | 29.9 ± 1.9 |

There was no great difference in the amount of L-lactic acid production after 24 hours between the condition of using calcium carbonate and that of using sodium hydroxide.

Under the condition of adding 5% (w/v) of calcium carbonate, when the concentration of lactic acid has risen to about 80 to 100 g/L, calcium lactate precipitated at 30° C. or 35° C., and further, the medium gelled. Since it gels in a state of mixture of cells and lactate, it is expected that the process of purifying L-lactic acid will be made complicated by this event. However, it is considered that this event can be avoided in the case of using sodium hydroxide, and so the availability of sodium hydroxide, depending on the purification method of L-lactic acid, may be a desirable characteristic in the process of producing L-lactic acid.

Twenty-four hours after the start of fermentation, the optical purity of L-lactic acid was higher with sodium hydroxide than with calcium carbonate.

Twenty-four hours after the start of fermentation, the amount of the by-product acetic acid was lower with sodium hydroxide than with calcium carbonate.

Furthermore, in addition to FIG. 13A which shows the results of two independent experiments, the results of three independent experiments are shown in FIG. 13B. According to the experimental data shown in FIG. 13B, the total sugar conversion rate after 24 hours of culture is 90.4 ±8.1%.

### Example 6

### Evaluation of the L-lactic Acid Production Ability of the Pj0957 Strain in Media with Sucrose as a Single Sugar Source

As shown below, evaluation of the lactic acid production ability of the Pj0957 strain was performed. The concentration of L-lactic acid in the medium was determined by using Biochemistry Analyzer (BA) from YSI JAPAN. An F-kit D-lactic acid/L-lactic acid from J.K. International was used to distinguish between optical isomers, and the procedure was carried out according to the attached protocol. The amounts of the production of other various organic acids were determined by using HPLC. Before used as a sample subjected to the analysis, the culture fluid was filtered through a 0.22 μm filter. The data are the average values of the results of at least three independent trials.

A loopful of a strain in pellet-like form, scraped using a platinum loop from yeast cells grown on YPD agar medium for 2 to 3 days at 30° C., was inoculated into 3 mL of YPD liquid medium contained in a 15 mL tube and pre-precultured for 20 to 30 hours at 30° C. in a benchtop culture apparatus from TAITEC at an amplitude of 35 mm at 130 rpm. This was inoculated into 100 mL of YPD medium contained in a Sakaguchi flask to an OD600 of about 0.1, and precultured at 30° C. in a benchtop culture apparatus from TAITEC at an amplitude of 35 mm at 130 rpm, typically for 15 to 22 hours. Then, the cells were collected by centrifugation under the conditions of 4° C., 3,000 rpm, and 5 minutes, and further, after removal of the supernatant, they were washed with the medium (not containing a neutralizing agent) used for fermentation.

The cells thus obtained were inoculated into a 15 mL volume of a medium containing 100 g/L sucrose in a 100 mL baffled Erlenmeyer flask, and fermented at 35° C. in a benchtop culture apparatus from TAITEC at an amplitude of 35 mm at 80 rpm. With regard to the amount of the cells inoculated for fermentation, the cells derived from the preculture were inoculated to an OD600 of 10, unless otherwise stated. Unless otherwise specified, calcium carbonate was added to the medium as a neutralizing agent to a concentration of 4.5% (w/v). The temperature during the fermentation was set at 30° C. or 35° C. Besides the above concentration of sucrose, 10 g/L yeast extract and 20 g/L peptone were added to the medium. The medium of this composition is hereinafter referred to as YPSuc10 medium.

The total sugar conversion rate (%) is a value obtained by dividing the weight of L-lactic acid in the medium by an initial weight of sucrose in the medium and then multiplying it by (342/360) and by 100. The optical purity (%) of L-lactic acid is a value obtained by dividing L-lactic acid concentration by L-lactic acid concentration plus D-lactic acid concentration and then multiplying it by 100.

As a result, for the amount of L-lactic acid production after 33 hours, the total sugar conversion rate was 94.8 ±3.5%, and the optical purity of L-lactic acid was greater than 99.9%. When the concentration of ethanol was quantitated by HPLC, it was found to be below the detection limit (less than 0.01 g/L).

[Sequence Listing]

1. A yeast strain of Candida utilis, wherein the yeast strain has been transformed with at least one copy of a gene that is operatively linked to a promoter sequence enabling expression of the gene and encodes a polypeptide having activity of lactate dehydrogenase.

2. The yeast strain according to claim 1, wherein an endogenous gene encoding a polypeptide having activity of pyruvate decarboxylase has been disrupted.

3. The yeast strain according to claim 1, wherein an endogenous gene encoding a polypeptide having activity of pyruvate decarboxylase has been disrupted by a deletion of the gene through an insertion of a selectable marker sequence.

4. The yeast strain according to claim 1, wherein the yeast strain has been transformed with an expression vector containing a promoter sequence and a DNA sequence encoding a polypeptide having activity of lactate dehydrogenase under control of the promoter sequence.

5. The yeast strain according to claim 1, wherein the polypeptide having the activity of lactate dehydrogenase is:
(a) a polypeptide comprising an amino acid sequence shown in SEQ ID NO:37; or
(b) a polypeptide comprising an amino acid sequence derived from the amino acid sequence shown in SEQ ID NO:37 by deletion, substitution, addition or insertion of one or several amino acids, and having the activity of lactate dehydrogenase.

6. The yeast strain according to claim 1, wherein the gene encoding a polypeptide having the activity of lactate dehydrogenase comprises:
(a) a nucleotide sequence from a at position 13 to a at position 1,011 of SEQ ID NO:36; or
(b) a nucleotide sequence having 85% or greater homology to the nucleotide sequence from a at position 13 to a at position 1,011 of SEQ ID NO:36, and encoding the polypeptide having the activity of lactate dehydrogenase; or
(c) a nucleotide sequence hybridizing with the nucleotide sequence from a at position 13 to a at position 1,011 of SEQ ID NO:36 or a sequence complementary thereto under stringent conditions, and encoding the polypeptide having the activity of lactate dehydrogenase.

7. The yeast strain according to claim 2, wherein the endogenous gene encoding a polypeptide having the activity of pyruvate decarboxylase comprises a nucleotide sequence encoding an amino acid sequence shown in SEQ ID NO:64, or a nucleotide sequence shown in SEQ ID NO:63.

8. The yeast strain according to claim 1, wherein the promoter sequence is a promoter region of the endogenous gene encoding pyruvate decarboxylase, or comprises a nucleotide sequence shown in SEQ ID NO:3.

9. A method for producing lactic acid, comprising culturing the yeast strain according to claim 1.

10. The method according to claim 9, wherein OD600 of cells at an initial stage of fermentation culture is 1 to 30 in the culturing of the yeast strain.


SEQUENCE LISTING (64 sequences)

SEQ ID NO: 1 - DNA, 26 bp, Artificial sequence
  Description primer IKSM-29
cargtyttrtggggttcyatyggttt

SEQ ID NO: 2 - DNA, 27 bp, Artificial sequence
  Description primer IKSM-30
ttcaatrgtgtarccmyygttgttcaa

SEQ ID NO: 3 - DNA, 218 bp, Candida utilis
tttcaatggtgtagccmyygttgttcaagacaaagatgtatggagtcaaaccccatctgatcatagtagagatctcttggacggtcaattgcaaggaaccatcaccgacgaacaagataactctcttctccttgtcaacttcctcagcagctgcaacggcacccaaagcagcaccaacagagaaaccratrgaaccccataaaacctgaagggcgaat

SEQ ID NO: 4 - DNA, 34 bp, Artificial sequence
  Description primer IM-135
gcggccgcatcgatgcaagataaacaagcaaggc

SEQ ID NO: 5 - DNA, 32 bp, Artificial sequence
  Description primer IM-136
gagctcggatccttggcaccaacaacctttgc

SEQ ID NO: 6 - DNA, 20 bp, Artificial sequence
  Description primer IM-19
caatcgatagattgtcgcac

SEQ ID NO: 7 - DNA, 20 bp, Artificial sequence
  Description primer IM-331
cgaaatgcatgcaagtaacc

SEQ ID NO: 8 - DNA, 20 bp, Artificial sequence
  Description primer IM-20
gcagtttcatttgatgctcg

SEQ ID NO: 9 - DNA, 20 bp, Artificial sequence
  Description primer IM-334
aagagtaagcattctggctc

SEQ ID NO: 10 - DNA, 20 bp, Artificial sequence
  Description primer IM-339
tatagtgcaggcgatcgtac

SEQ ID NO: 11 - DNA, 20 bp, Artificial sequence
  Description primer IM-340
cagtgatgtcggaaacatcg

SEQ ID NO: 12 - DNA, 39 bp, Artificial sequence
  Description primer IM-147
tttgattgatttgactgtgttattttgcgtgaggttatg

SEQ ID NO: 13 - DNA, 53 bp, Artificial sequence
  Description primer IM-150
gcgatttaatctctaattattagttaaagttttataagcatttttatgtaacg

SEQ ID NO: 14 - DNA, 65 bp, Artificial sequence
  Description primer IM-148
tctactcataacctcacgcaaaataacacagtcaaatcaatcaaaatgagcgaaatcacattggg

SEQ ID NO: 15 - DNA, 77 bp, Artificial sequence
  Description primer IM-149
cgttacataaaaatgcttataaaactttaactaataattagagattaaatcgcttattgctgcttgttggtgttggc

SEQ ID NO: 16 - DNA, 99 bp, Artificial sequence
  Description primer IM-53
cggccgccagctgaagcttcgtacgctgcaggtcgacaacccttaatataacttcgtataatgtatgctatacgaagttataccagaggacacgtaacc

SEQ ID NO: 17 - DNA, 68 bp, Artificial sequence
  Description primer IM-57
catgaggatcataatttataacgtaatcccataaataaaagtcatacaatcattcctttgccctcgga

SEQ ID NO: 18 - DNA, 34 bp, Artificial sequence
  Description loxP sequence
ataacttcgtataatgtatgctatacgaagttat

SEQ ID NO: 19 - DNA, 50 bp, Artificial sequence
  Description primer IM-54
attgtatgacttttatttatgggattacgttataaattatgatcctcatg

SEQ ID NO: 20 - DNA, 83 bp, Artificial sequence
  Description primer IM-55
taggccactagtggatctgatatcacctaataacttcgtatagcatacattatacgaagttattcattcatccctcactatcg

SEQ ID NO: 21 - DNA, 20 bp, Artificial sequence
  Description primer IM-1
ggccgccagctgaagcttcg

SEQ ID NO: 22 - DNA, 20 bp, Artificial sequence
  Description primer IM-2
aggccactagtggatctgat

SEQ ID NO: 23 - DNA, 40 bp, Artificial sequence
  Description primer IM-49
ctcggtacctctagaatgtccaatttactgaccgtacacc

SEQ ID NO: 24 - DNA, 40 bp, Artificial sequence
  Description primer IM-50
gcatcaacgttttcttttcgtatgcgccgcataaccagtg

SEQ ID NO: 25 - DNA, 40 bp, Artificial sequence
  Description primer IM-51
cactggttatgcggcgcatacgaaaagaaaacgttgatgc

SEQ ID NO: 26 - DNA, 37 bp, Artificial sequence
  Description primer IM-52
ctcggtaccggatccctaatcgccatcttccagcagg

SEQ ID NO: 27 - DNA, 20 bp, Artificial sequence
  Description primer IM-277
agttggactcggatcatctc

SEQ ID NO: 28 - DNA, 50 bp, Artificial sequence
  Description primer IM-278
ctgcagcgtacgaagcttcagctggcggccaacattcctacgctcagagc

SEQ ID NO: 29 - DNA, 50 bp, Artificial sequence
  Description primer IM-279
attaggtgatatcagatccactagtggcctacagaacgctcctaaacacg

SEQ ID NO: 30 - DNA, 20 bp, Artificial sequence
  Description primer IM-280
gaacttctccaacaggtagc

SEQ ID NO: 31 - DNA, 20 bp, Artificial sequence
  Description primer IM-281
cacattggaaccatatgatc

SEQ ID NO: 32 - DNA, 20 bp, Artificial sequence
  Description primer IM-282
gagctcatcaaagtacaagg

SEQ ID NO: 33 - DNA, 50 bp, Artificial sequence
  Description primer IM-185
attaggtgatatcagatccactagtggccttgcaattgaccgtccaagag

SEQ ID NO: 34 - DNA, 20 bp, Artificial sequence
  Description primer IM-168
tcttgtacgtgtttaggagc

SEQ ID NO: 35 - protein, 332 aa, Bos taurus
MATLKDQLIQNLLKEEHVPQNKITIVGVGAVGMACAISILMKDLADEVALVDVMEDKLKGEMMDLQHGSLFLRTPKIVSGKDYNVTANSRLVIITAGARQQEGESRLNLVQRNVNIFKFIIPNIVKYSPNCKLLVVSNPVDILTYVAWKISGFPKNRVIGSGCNLDSARFRYLMGERLGVHPLSCHGWILGEHGDSSVPVWSGVNVAGVSLKNLHPELGTDADKEQWKAVHKQVVDSAYEVIKLKGYTSWAIGLSVADLAESIMKNLRRVHPISTMIKGLYGIKEDVFLSVPCILGQNGISDVVKVTLTHEEEACLKKSADTLWGIQKELQF

SEQ ID NO: 36 - DNA, 1026 bp, Artificial sequence
  Description Bos taurus L-LDH-A gene, whose codon-usage was optimized for Candida utilis
ggtacctctagaatggctaccttgaaggaccaattgatccagaacttgttgaaggaagagcacgttccacaaaacaagatcaccatcgttggtgttggtgctgttggcatggcttgtgccatctccatcttgatgaaggatttggctgatgaggttgccttggttgacgtcatggaagataagttgaagggtgaaatgatggacttgcagcacggttctttgttcttgagaaccccaaagatcgtttccggcaaggactacaacgttaccgctaactccagattggttatcatcaccgctggtgctagacaacaagagggtgagtccagattgaacttggtccagagaaacgtcaacatcttcaagttcatcatcccaaacatcgtcaagtactccccaaactgcaagttgttggttgtctccaacccagttgacatcttgacctacgttgcttggaagatttctggtttcccaaagaacagagtcatcggttccggttgtaacttggactccgccagattcagatacttgatgggtgaaagattgggtgttcacccattgtcttgtcacggctggatcttgggtgaacacggtgattcttccgttccagtttggtccggtgttaacgttgctggtgtctccttgaagaacttgcacccagagttgggtacagatgctgacaaggaacaatggaaggctgttcacaagcaagttgttgactccgcttacgaggtcatcaagctgaagggctacacctcttgggctatcggtttgtctgttgctgatttggctgagtccatcatgaagaacttgagaagagtccacccaatctccaccatgatcaagggtttgtacggtatcaaggaagatgttttcttgtccgtcccatgtatcttgggtcaaaacggtatctccgacgttgttaaggttaccttgacccacgaagaagaggcttgtttgaagaagtctgccgataccttgtggggtatccagaaggaattgcagttctgatgaggatccgagctc

SEQ ID NO: 37 - protein, 332 aa, Artificial sequence; identical to SEQ ID NO: 35
  Description Bos taurus L-LDH-A gene, whose codon-usage was optimized for Candida utilis

SEQ ID NO: 38 - DNA, 999 bp, Bos taurus
atggcaactctcaaggatcagctgattcagaatcttcttaaggaagaacatgtcccccagaataagattacaattgttggggttggtgctgttggcatggcctgtgccatcagtatcttaatgaaggacttggcagatgaagttgctcttgttgatgtcatggaagataaactgaagggagagatgatggatctccaacatggcagccttttccttagaacaccaaaaattgtctctggcaaagactataatgtgacagcaaactccaggctggttattatcacagctggggcacgtcagcaagagggagagagccgtctgaatttggtccagcgtaacgtgaacatctttaaattcatcattcctaatattgtaaaatacagcccaaattgcaagttgcttgttgtttccaatccagtcgatattttgacctatgtggcttggaagataagtggctttcccaaaaaccgtgttattggaagtggttgcaatctggattcagctcgcttccgttatctcatgggggagaggctgggagttcacccattaagctgccatgggtggatccttggggagcatggtgactctagtgtgcctgtatggagtggagtgaatgttgctggtgtctccctgaagaatttacaccctgaattaggcactgatgcagataaggaacagtggaaagcggttcacaaacaagtggttgacagtgcttatgaggtgatcaaactgaaaggctacacatcctgggccattggactgtcagtggccgatttggcagaaagtataatgaagaatcttaggcgggtgcatccgatttccaccatgattaagggtctctatggaataaaagaggatgtcttccttagtgttccttgcatcttgggacagaatggaatctcagacgttgtgaaagtgactctgactcatgaagaagaggcctgtttgaagaagagtgcagatacactttgggggatccagaaagaactgcagttttaa

SEQ ID NO: 39 - DNA, 68 bp, Artificial sequence
  Description primer IM-345
actcgcgcgcaagatctaagcggccgctaatggatccaataatcgatgctgtctttcttcttcatggg

SEQ ID NO: 40 - DNA, 37 bp, Artificial sequence
  Description primer IM-346
actcgcgcgcaagatctgaacttctccaacaggtagc

SEQ ID NO: 41 - DNA, 99 bp, Artificial sequence
  Description primer IM-283
cggccgccagctgaagcttcgtacgctgcaggtcgacaacccttaatataacttcgtataatgtatgctatacgaagttatcctttgctgtgttctacc

SEQ ID NO: 42 - DNA, 90 bp, Artificial sequence
  Description primer IM-349
acaactattccaatccacgatggaaactgacaatcttgaatcatagacgccaacagtgtgaagagtctccagctgaagcttcgtacgctg

SEQ ID NO: 43 - DNA, 40 bp, Artificial sequence
  Description primer IM-350
actcggccggccatcgatcactagtggatctgatatcacc

SEQ ID NO: 44 - DNA, 44 bp, Artificial sequence
  Description primer IM-347
actcggatccctgcaagctactttgtaattaaacaaataacggg

SEQ ID NO: 45 - DNA, 80 bp, Artificial sequence
  Description primer IM-348
ggagactcttcacactgttggcgtctatgattcaagattgtcagtttccatcgtggattggaatagttgtggtgaccttg

SEQ ID NO: 46 - DNA, 38 bp, Artificial sequence
  Description primer IM-341
actcgcggccgctctagacaccaactttgaagataggg

SEQ ID NO: 47 - DNA, 64 bp, Artificial sequence
  Description primer IM-342
tggtccttcaaggtagccatggtatcgattgttttagttttgtttgtttgttgtgtataacggg

SEQ ID NO: 48 - DNA, 64 bp, Artificial sequence
  Description primer IM-343
cccgttatacacaacaaacaaacaaaactaaaacaatcgataccatggctaccttgaaggacca

SEQ ID NO: 49 - DNA, 35 bp, Artificial sequence
  Description primer IM-379
actcagatcttcatcagaactgcaattccttctgg

SEQ ID NO: 50 - DNA, 20 bp, Artificial sequence
  Description primer IM-362
caccttcaatagcgaagttc

SEQ ID NO: 51 - DNA, 20 bp, Artificial sequence
  Description primer IM-174
gagctcatcaaagtacaagg

SEQ ID NO: 52 - DNA, 20 bp, Artificial sequence
  Description primer IM-163
gattgatgctactcagttcc

SEQ ID NO: 53 - DNA, 20 bp, Artificial sequence
  Description primer IM-164
aaggcagaggtaccagtctc

SEQ ID NO: 54 - DNA, 20 bp, Artificial sequence
  Description primer IM-59
aagagagcacaagacgatgg

SEQ ID NO: 55 - DNA, 50 bp, Artificial sequence
  Description primer IM-60
ctgcagcgtacgaagcttcagctggcggcctactctcgttaaacagctcg

SEQ ID NO: 56 - DNA, 50 bp, Artificial sequence
  Description primer IM-61
attaggtgatatcagatccactagtggcctattggattcatcgcacagag

SEQ ID NO: 57 - DNA, 20 bp, Artificial sequence
  Description primer IM-62
tcctttctggattggattgg

SEQ ID NO: 58 - DNA, 20 bp, Artificial sequence
  Description primer IM-63
aagcttatggaggagattgg

SEQ ID NO: 59 - DNA, 20 bp, Artificial sequence
  Description primer IM-92
cagaacttgagtcaatcacc

SEQ ID NO: 60 - DNA, 20 bp, Artificial sequence
  Description primer IM-223
tttcaggcaggtcttgcaac

SEQ ID NO: 61 - DNA, 20 bp, Artificial sequence
  Description primer IM-295
taggatacaggctagagatc

SEQ ID NO: 62 - DNA, 50 bp, Artificial sequence
  Description primer IM-296
ctgcagcgtacgaagcttcagctggcggcccattcatagctgggcttctc

SEQ ID NO: 63 - DNA, 5014 bp, Candida utilis
  CDS (2247)..(3938)
acaggttcctcttcaccggtcaattcattcaactttttctcaggttgcggttccaagtatagcgcaatgttgttatttgcgttccccttggggaaaagtaatatgtcaaattcccaatcaccaactttgaagatagggcttcggactttatcctcaaccaacttcttatagtcattgatctcccaggtgtaagctccgtaatgtagcacctcctcgtcatcaatctttggtaacacttggtctttcaagaaggcgaaatcaatggacgtgccggactctaatagttgctccttccttgtgtcaggcaccaagtttggctctggtaatggttcgctatttgtagcttcaacgtcagcgtcgctcatttcatccactgtgttctctttccaattctatgccgatgatctcaattgtaagatttgccacttctctgcgcctatttcctgtttatctttacttgttttgactgtttattctgttttggtttctgggtttttgtgatgctgtttgcctttttttgcccttttcccttttgcgtatccttctaaacttggaaaggggggttttatagaaaaccaaatcttcaccttacttaaagtgacacaagttgtaaaaagaaaggacacacacaaactccaattcaagtagctgctttgccttgtaaaattgaatggaaattggacgatcgtgcctgtgtttccggtcttccactaacaactggatcctttgtcaccaaaatgaaaagccaaaatggagagattgttgatttctttgtttaatctatgacacacagagaacgttgcgcgcgcaacgaagaatacgccagagacggataacagatatagattatgtaatttgttacgtaagtagttacgaaagcaagtcttgactcttggggatcgagcctccgaacctgttgtagaatttcgtggggtgtattgccatcggaaaaactatacggttgttgacgtggggggctcgagatgaggctcaagtttcaagtttcaaccgtttcaactggttcaatctttgccactgagcgaaaacatgggcagaagcgatcaaaaaggagaaaaagaaaatcatcaagtggtacccgcgcgcacttattgtttgatgcatttagattatcaatcatacgaaagcaactgtacaccacagatcacccatggctcaaacacacaacctaatattaggaagaaaaaataaagagctccgatcctccacattggaaccatatgatcgataacgcacataccatccgctacaatccatgtaaactctcccccgtgtaaactctctccgtgctatactgcaagacacacacactgttgctccagttgaagcggaagttggactcggatcatctcctgggtttgaacatcaagcacctggaaaccaggaaacctctctgcagcttgctgtctagaactaacacccacatccctgctcatcgtgattcgctctggttcgcccattgctgcctcgcacgcttttactatgcaaccgcatcaatgcctggttattcgttttcgatacgtgcttaagcccaggggatcatcaccagttgacaccaagcaaaaaagaaacaatcgcctcgctggcggaaattagaaaacaacgcaggtgtgcgaattctcacgatatatcgtacaagccattgcacaccgcacgtctgtgcagtttgccatcaatcgacgtttatagcacacaccatagatcacacagagcttcggctctattccgttccgtagagaaaagcaaccattgtatacaaacaccaaggctatcgcttcccgattcgtcaaatgcacagcttcccttcaggtggcccagctgagcgcaacatgaaaaccggtcctgcgcacactttatccaagcatgtggatggcaccatgcggttaaaaactatgcacttatgcaacgatacgggcacgatttgaatgagttgaacacacacacgcgtagcacacactggtttatgcctggttaagcagtgccctctgtattcgctgtctgcgtcaaatggtgtcaaaaggactaaaaggaatttttaggaaagggggagtggtcagttggtaccgttgtcgtcttacatatataagaagctagggtttcccactttgctctgagcgtaggaatgtttcaactcatcatcatctattacaggaaactcaattgaatcatcattttcaattgatacccgttatacacaacaaacaaacaaaactaaaacaatcgataccatgagcgaaatcacattgggacgttacctcttcgagagactcaaacaagttgaggtcaacaccgtcttcggtcttccaggtgacttcaacctttgtctcttggataagctctacgaagttgacggcatgagatgggctggtaacgccaacgagttgaacgccgcctacgccgctgatggttactccagagttaagaagcttgctgctatcatcaccactttcggtgtcggtgagttgtccgccttgaacggtattgccggttcttactctgagcacgttggtttgctccacattgttggtgtcccatctatctcatcccaggcaaagcaattgcttttgcaccacactttgggtaacggtgacttcacagtcttccacagaatgtcatcaaacatttcccaaacaaccgctttcatcaaggacatcaactctgctgcctctgaaatcgaccgttgtatcagaaccgcttacgtctaccaaagaccagtgtaccttgcccttccagcaaacttggttgacgatttggttccagcctcccttttgaacacaccaattgatttgtcattgaagccaaacgacccagaagctgaggatgaggtcatccaaaccgtttgtgagatggttcaaaaggctaagaacccagtcatcttggttgatgcctgtgcctctcgtcacgatgtcaagaaggagacaaaggatttgattgatgctactcagttcccagcctttgtcacacctatgggtaagggtggtgtcgatgaacaacacccaagattcggtggtgtttacgtcggtactttgtctaagccagatgtcaaggaggctgtcgagtctgctgacttggttttgtctgtcggtgccatcttgtccgatttcaacaccggttctttctcttactcctacaagaccaacaacattgttgaattccactctgattacatcaagatcaagaacgccaccttcccaggtgtccaattcaagtttgtcttgcaaaagcttgtcaaggccattaagccattcgtcaaggactacacaccagttccagttccaactttgaagttgatcaactctccacactctccacaaacccctttgactcaagaatgggtctggaccaagttgtcctcatggttgcgtgaaggtgatgtcgtcatcactgagactggtacctctgccttcggtatcgtccaaaccagattcccaaacaacaccactggtatctcccaagtcttgtggggttctattggttactctgttggtgctgctttgggtgccgttgcagctgctgaggaagttgacaaggagaagagagttatcttgttcgtcggtgatggttccttgcaattgaccgtccaagagatctctactatgatcagatggggtttgactccatacatctttgtcttgaacaacgatggttacaccattgagagattgatccacggtgaaaaggctggttacaacgatatccagaactgggaccacttggctctcttgccaaccttcggtgctaaagtgtacgacagcatcagagtctccactacaggtgagtttgagcaattgactcaaagtgctgagtttgccaagaactccaagattagattgattgaggtcatgttgccaactatggatgctccattgaacttggtcaagcaagcccaattgactgccaacaccaacaagcagcaataaaaggaaaccggagcaggaccgggctgtctttcttcttcatgggtttagttagttatatataagttttcatttaattgctttaatttgttttatcaatgctgtattgagtcttttataacaacagaacgctcctaaacacgtacaagacagccatcgcgtccaacaaggagagcctgacacacacgcatgcttcctcaagcggctttcattggttgtctctaaaatgaaatttgtgccccctacttgaacaattctcccgtgtacgcgttctagttaaccatttttttatgttttctgccgtccactttgatctacgttgaagttaaggaacctttacattgttggatttagccccttacgctccctttgtttcacactactagagctggcgctgctgaagatcctattcaatccatattgatccagaaacgataaccgcatagcgagcgctgaaagagctggaaaaagatagaggagaaaacacagctttgttcttcgcttgagacaaagacataagtaactggtgctttcacgagactgcttgggggttattgttgctgtagttttgttgtctactcgagctcgctccgtttcgtcatttaccgctgtttttaactttgcaatagtccgtgtgtgtgtgtgttcgtattaacagttaatgtgtggaaaatggttcctggaatcaacaagcagaagctcgtgaatacaacgagacagtatttacacgctgtgaacatgaatttctccttcaaccaggtgaaatacatcatgaccttggtggcactgttcgttgtgctttgtcaactgctgataatggcatatccgtcatctagtcccgtcagcttgcagaccatgactagtgggactgctcaggacctcgtgttggtgttggatgagaacatgccggtctcaacatattatggctacctgttggagaagttctccgagaactttccttcgaagccgcttgaggagaaatgtgccttgtactttgatgagctctacaagagggatgagaactgggaagttatggaccctgagagtaatgcggataaggactatcagatggactcata

SEQ ID NO: 64 - protein, 563 aa, Candida utilis
MSEITLGRYLFERLKQVEVNTVFGLPGDFNLCLLDKLYEVDGMRWAGNANELNAAYAADGYSRVKKLAAIITTFGVGELSALNGIAGSYSEHVGLLHIVGVPSISSQAKQLLLHHTLGNGDFTVFHRMSSNISQTTAFIKDINSAASEIDRCIRTAYVYQRPVYLALPANLVDDLVPASLLNTPIDLSLKPNDPEAEDEVIQTVCEMVQKAKNPVILVDACASRHDVKKETKDLIDATQFPAFVTPMGKGGVDEQHPRFGGVYVGTLSKPDVKEAVESADLVLSVGAILSDFNTGSFSYSYKTNNIVEFHSDYIKIKNATFPGVQFKFVLQKLVKAIKPFVKDYTPVPVPTLKLINSPHSPQTPLTQEWVWTKLSSWLREGDVVITETGTSAFGIVQTRFPNNTTGISQVLWGSIGYSVGAALGAVAAAEEVDKEKRVILFVGDGSLQLTVQEISTMIRWGLTPYIFVLNNDGYTIERLIHGEKAGYNDIQNWDHLALLPTFGAKVYDSIRVSTTGEFEQLTQSAEFAKNSKIRLIEVMLPTMDAPLNLVKQAQLTANTNKQQ